(19) United States
(12) Patent Application Publication
(10) Pub. No.: US 2025/0120714 A1
(43) Pub. Date: Apr. 17, 2025

(54) DUAL INDEPENDENT KEYED LOCKING MEMBERS ACTING ON THE SAME DRIVE MEMBER

(71) Applicant: Cilag GmbH International, Zug (CH)

(72) Inventors: Frederick E. Shelton, IV, Hillsboro, OH (US); Jason L. Harris, Lebanon, OH (US)

(21) Appl. No.: 18/379,817

(22) Filed: Oct. 13, 2023

(51) Int. Cl. A61B 17/072

(52) U.S. Cl. CPC A61B 17/07207; A61B 2017/07271

(57) ABSTRACT

A surgical stapling system is disclosed that includes a firing drive, a first lockout, and a second lockout. The firing drive comprises a first edge and a second edge. The first lockout is configurable between a first locked state in which the first lockout engages the first edge during the firing stroke and a first unlocked state in which the first edge bypasses the first lockout during the firing stroke. The second lockout is configurable between a second locked state in which the second lockout engages the second edge during the firing stroke and a second unlocked state in which the second edge bypasses the second lockout during the firing stroke. A staple cartridge is seatable in the stapling system that comprises a first key that unlocks the first lockout and a second key that unlocks the second lockout.

### BACKGROUND

The present invention relates to surgical instruments and, in various arrangements, to surgical stapling and cutting instruments and staple cartridges for use therewith that are designed to staple and cut tissue.

### SUMMARY

Lockouts are utilized with surgical stapling and cutting instruments to ensure that certain conditions are met prior to allowing a clinician to staple and cut patient tissue with the surgical stapling and cutting instrument. These lockouts ensure that patient tissue is not inadvertently cut and/or stapled prior to these conditions being met.

In accordance with the present disclosure, a surgical stapling system comprises an end effector, a firing actuator, a first lockout, and a second lockout. The end effector comprises a first jaw and a second jaw pivotable relative to the first jaw between an open position and a closed position. The firing actuator is movable from a proximal position toward a distal position during a firing stroke. The firing stroke comprises a first stroke portion and a second stroke portion distal to the first stroke portion. The firing actuator comprises a first edge and a second edge. The first lockout is configurable in a first locked state in which the first lockout engages the first edge during the firing stroke and a first unlocked state in which the first edge bypasses the first lockout during the firing stroke. The second lockout is configurable in a second locked state in which the second lockout engages the second edge during the firing stroke and a second unlocked state in which the second edge bypasses the second lockout during the firing stroke. A staple cartridge comprising a first key is configured to place the first lockout in the first unlocked state based on the staple cartridge being positioned in the end effector. A staple cartridge comprising a second key is configured to place the second lockout in the second unlocked state based on the staple cartridge being positioned in the end effector.

### LISTING OF THE FIGURES

Various features of the embodiments described herein, together with advantages thereof, may be understood in accordance with the following description taken in conjunction with the accompanying drawings as follows:

FIG. 1 is a partial perspective view of a surgical stapling system including an end effector;

FIG. 2 is a perspective view of the end effector of FIG. 1 with a staple cartridge removed therefrom;

FIG. 3 is another perspective view of the end effector of FIG. 1 with the staple cartridge removed;

FIG. 4 is an elevational view of the end effector of FIG. 1 in a closed position;

FIG. 5 is an elevational view of the end effector of FIG. 1 in an open position;

FIG. 6 illustrates a lockout system comprising a first lockout and a second lockout for using with the surgical stapling system of 1;

FIG. 7 illustrates a top view of the lockout system of FIG. 6 with a compatible staple cartridge being inserted into the end effector;

FIG. 8 illustrates the lockout system of FIG. 6 with the staple cartridge of FIG. 7 transitioning the lockout system to an unlocked state;

FIG. 9 illustrates the lockout system of FIG. 6 with a staple cartridge unlocking the first lockout but not the second lockout;

FIG. 10 illustrates the lockout system of FIG. 6 with a staple cartridge unlocking the second lockout but not the first lockout;

FIG. 11 illustrates the lockout system of FIG. 6 and a firing driver having longitudinally staggered lock shoulders;

FIG. 12 illustrates the system of FIG. 11 with a staple cartridge positioned in the end effector that has unlocked both the first lockout and the second lockout of the lockout system of FIG. 6;

FIG. 13 illustrates the system of FIG. 12 with the firing driver moving through the staple cartridge;

FIG. 14 illustrates the system of FIG. 11 with a staple cartridge unlocking the second lockout of the lockout system but not the first lockout;

FIG. 15 illustrates the system of FIG. 11 with a staple cartridge unlocking the first lockout of the lockout system but not the second lockout;

FIG. 16 illustrates the system of FIG. 11 and the staple cartridge of FIG. 15 with the lockout system preventing distal motion of the firing driver after a first portion of its stroke;

FIG. 17 illustrates the lockout system of FIG. 6 with a firing driver having an elongate lock window;

FIG. 18 illustrates the system of FIG. 17 with a compatible staple cartridge positioned in the end effector that has unlocked the first lockout and the second lockout of the lockout system;

FIG. 19 illustrates the system of FIG. 18 with the firing driver moving through the staple cartridge;

FIG. 20 illustrates the system of FIG. 17 with a staple cartridge that has unlocked the second lockout of the lockout system but not the first lockout;

FIG. 21 illustrates the system of FIG. 17 with a staple cartridge that has unlocked the first lockout of the lockout system but not the second lockout;

FIG. 22 illustrates the system of FIG. 17 and the staple cartridge of FIG. 21 with the lockout system preventing distal motion of the firing driver after a first portion of its stroke;

FIG. 23 illustrates a lockout system and a firing actuator for use with the surgical stapling system of FIG. 1; and

FIG. 24 illustrates the lockout system of FIG. 23 in a locked configuration.

Corresponding reference characters indicate corresponding parts throughout the several views.

### DETAILED DESCRIPTION

Applicant of the present application owns the following U.S. Patent Applications that were filed on even date herewith and which are each herein incorporated by reference in their respective entireties:
U.S. Patent Application, titled METHOD OF OPERATING A SURGICAL STAPLING INSTRUMENT; Attorney Docket No. END9484USNP2/220491-2M;
U.S. Patent Application, titled SURGICAL STAPLING SYSTEMS WITH ADAPTIVE STAPLE FIRING ALGORITHMS; Attorney Docket No. END9484USNP3/220491-3;
U.S. Patent Application, titled LEARNED TRIGGERS FOR ADAPTIVE CONTROL OF SURGICAL STAPLING SYSTEMS; Attorney Docket No. END9484USNP4/220491-4;
U.S. Patent Application, titled CONTROL CIRCUIT FOR ACTUATING MOTORIZED FUNCTION OF SURGICAL STAPLING INSTRUMENT UTILIZING INERTIAL DRIVE TRAIN PROPERTIES; Attorney Docket No. END9484USNP5/220491-5;
U.S. Patent Application, titled PROPORTIONATE BALANCING OF THE FUNCTION IMPACT MAGNITUDE OF BATTERY OUTPUT TO PEAK MOTOR CURRENT; Attorney Docket No. END9484USNP6/220491-6;
U.S. Patent Application, titled MOTOR OPTIMIZATION BY MINIMIZATION OF PARASITIC LOSSES AND TUNING MOTOR DRIVE CONFIGURATION; Attorney Docket No. END9484USNP7/220491-7;
U.S. Patent Application, titled APPARATUS AND METHOD TO REDUCE PARASITIC LOSSES OF THE ELECTRICAL SYSTEM OF A SURGICAL INSTRUMENT; Attorney Docket No. END9484USNP8/220491-8;
U.S. Patent Application, titled SURGICAL TOOL WITH RELAXED FLEX CIRCUIT ARTICULATION; Attorney Docket No. END9484USNP9/220491-9;
U.S. Patent Application, titled WIRING HARNESS FOR SMART STAPLER WITH MULTI AXIS ARTICULATION; Attorney Docket No. END9484USNP10/220491-10;
U.S. Patent Application, titled SURGICAL SYSTEM WITH WIRELESS ARRAY FOR POWER AND DATA TRANSFER; Attorney Docket No. END9484USNP11/220491-11; and.
U.S. Patent Application, titled SURGICAL STAPLE CARTRIDGE COMPRISING REPLACEABLE ELECTRONICS PACKAGE; Attorney Docket No. END9484USNP12/220491-12.

Applicant of the present application owns the following U.S. Patent Applications that were filed on even date herewith and which are each herein incorporated by reference in their respective entireties:
U.S. Patent Application, titled METHOD OF ASSEMBLING A STAPLE CARTRIDGE; Attorney Docket No. END9484USNP13/220491-13M;
U.S. Patent Application, titled CONTROL SURFACES ON A STAPLE DRIVER OF A SURGICAL STAPLE CARTRIDGE; Attorney Docket No. END9484USNP14/220491-14;
U.S. Patent Application, titled INTEGRAL CARTRIDGE STIFFENING FEATURES TO REDUCE CARTRIDGE DEFLECTION; Attorney Docket No. END9484USNP15/220491-15;
U.S. Patent Application, titled STAPLE CARTRIDGE COMPRISING WALL STRUCTURES TO REDUCE CARTRIDGE DEFLECTION; Attorney Docket No. END9484USNP16/220491-16;
U.S. Patent Application, titled PAN-LESS STAPLE CARTRIDGE ASSEMBLY COMPRISING RETENTION FEATURES FOR HOLDING STAPLE DRIVERS AND SLED; Attorney Docket No. END9484USNP17/220491-17;
U.S. Patent Application, titled STAPLE CARTRIDGE COMPRISING A SLED HAVING A DRIVER LIFT CAM; Attorney Docket No. END9484USNP18/220491-18;
U.S. Patent Application, titled SURGICAL STAPLE CARTRIDGES WITH SLEDS CONFIGURED TO BE COUPLED TO A FIRING DRIVER OF A COMPATIBLE SURGICAL STAPLER; Attorney Docket No. END9484USNP19/220491-19;
U.S. Patent Application, titled STAPLE CARTRIDGE COMPRISING A COMPOSITE SLED; Attorney Docket No. END9484USNP20/220491-20;
U.S. Patent Application, titled SURGICAL INSTRUMENTS WITH JAW AND FIRING ACTUATOR LOCKOUT ARRANGEMENTS LOCATED PROXIMAL TO A JAW PIVOT LOCATION; Attorney Docket No. END9484USNP21/220491-21;
U.S. Patent Application, titled SURGICAL INSTRUMENTS WITH LATERALLY ENGAGEABLE LOCKING ARRANGEMENTS FOR LOCKING A FIRING ACTUATOR; Attorney Docket No. END9484USNP22/220491-22;
U.S. Patent Application, titled ADJUNCTS FOR USE WITH SURGICAL STAPLING INSTRUMENTS; Attorney Docket No. END9484USNP24/220491-24;
U.S. Patent Application, titled ADJUNCTS FOR USE WITH SURGICAL STAPLING INSTRUMENTS; Attorney Docket No. END9484USNP25/220491-25;
U.S. Patent Application, titled JAW CONTROL SURFACES ON A SURGICAL INSTRUMENT JAW; Attorney Docket No. END9484USNP26/220491-26;
U.S. Patent Application, titled ZONED ALGORITHM ADAPTIVE CHANGES BASED ON CORRELATION OF COOPERATIVE COMPRESSION CONTRIBUTIONS OF TISSUE; Attorney Docket No. END9484USNP27/220491-27;
U.S. Patent Application, titled STAPLE CARTRIDGES COMPRISING TRACE RETENTION FEATURES; Attorney Docket No. END9484USNP29/220491-29; and
U.S. Patent Application, titled STAPLE CARTRIDGES COMPRISING STAPLE RETENTION FEATURES; Attorney Docket No. END9484USNP30/220491-30.

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the embodiments described and illustrated herein are non-limiting examples, and thus it can be appreciated that the specific structural and functional details disclosed herein may be representative and illustrative. Variations and changes thereto may be made without departing from the scope of the claims.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Various exemplary devices and methods are provided for performing laparoscopic and minimally invasive surgical procedures. However, the reader will readily appreciate that the various methods and devices disclosed herein can be used in numerous surgical procedures and applications including, for example, in connection with open surgical procedures. As the present Detailed Description proceeds, the reader will further appreciate that the various instruments disclosed herein can be inserted into a body in any way, such as through a natural orifice, through an incision or puncture hole formed in tissue, etc. The working portions or end effector portions of the instruments can be inserted directly into a patient's body or can be inserted through an access device that has a working channel through which the end effector and elongate shaft of a surgical instrument can be advanced.

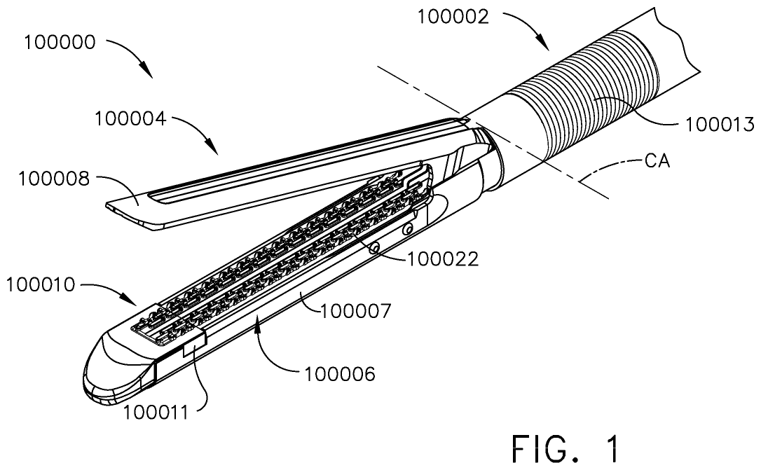

Referring to FIG. 1, a surgical stapling system 100000 comprises a shaft 100002 and an end effector 100004 extending from the shaft 100002. The end effector 100004 comprises a first jaw 100006 and a second jaw 100008. The first jaw 100006 comprises an elongate channel 100007 and a staple cartridge 100010. The staple cartridge 100010 is configured to be replaced during a surgical procedure. The staple cartridge 100010 comprises tabs 100011 on the lateral sides thereof that fit within corresponding recesses 100009 defined in the elongate channel 100007 and align the staple cartridge 100010 in the channel 100007. The staple cartridge 100010 comprises one or more lock shoulders that releasably engage the elongate channel 100007 and permit the staple cartridge 100010 to be removed from the channel 100007. Alternatively, the staple cartridge 100010 may not be removable from, or at least readily replaceable from, the first jaw 100006.

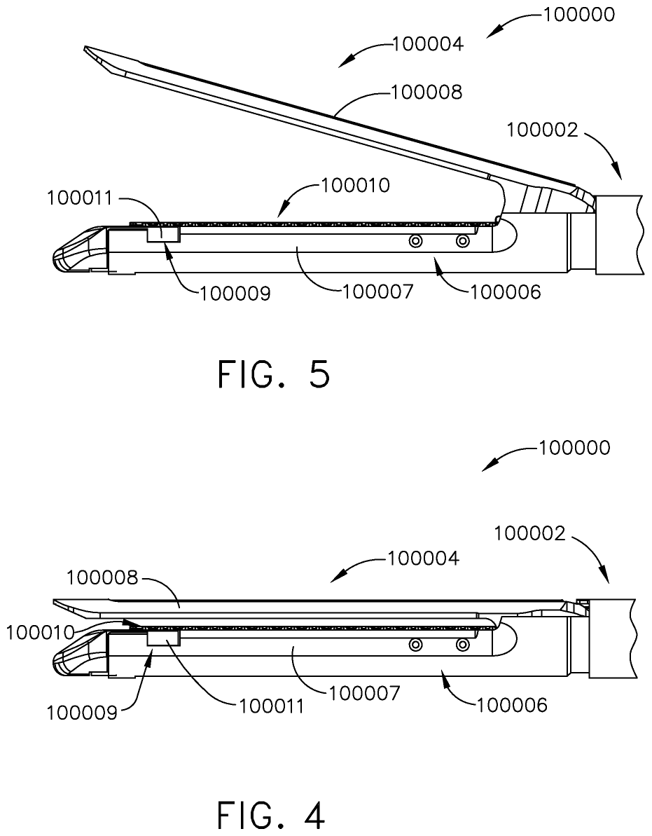

The second jaw 100008 comprises an anvil configured to deform staples ejected from the staple cartridge 100010. The second jaw 100008 is pivotable relative to the first jaw 10006 about a closure axis CA between an open position (FIG. 5) and a closed position (FIG. 4); however, the first jaw 100006 may be pivotable relative to the second jaw 100008. The surgical stapling system 100000 further comprises an articulation joint 100013 configured to permit the end effector 100004 to be rotated, or articulated, relative to the shaft 100002. However, the surgical stapling system 1000000 may not include an articulation joint.

The staple cartridge 100010 comprises a cartridge body 100012. The cartridge body 100012 includes a proximal end 100014, a distal end 100016, and a deck 100018 extending between the proximal end 100014 and the distal end 100016. In use, the staple cartridge 100010 is positioned on a first side of the tissue to be stapled and the anvil 100008 is positioned on a second side of the tissue. The anvil 100008 is moved toward the staple cartridge 100010 (see FIGS. 4 and 5) to compress and clamp the tissue against the deck 100018. Thereafter, staples 100021 removably stored in the cartridge body 100012 can be deployed into the tissue. The cartridge body 100012 includes staple cavities 100020 defined therein wherein staples are removably stored in the staple cavities 100020. The staple cavities 100020 are arranged in six longitudinal rows. Three rows of staple cavities are positioned on a first side of a longitudinal slot 100022 and three rows of staple cavities are positioned on a second side of the longitudinal slot 100022. Other arrangements of staple cavities and staples may be possible.

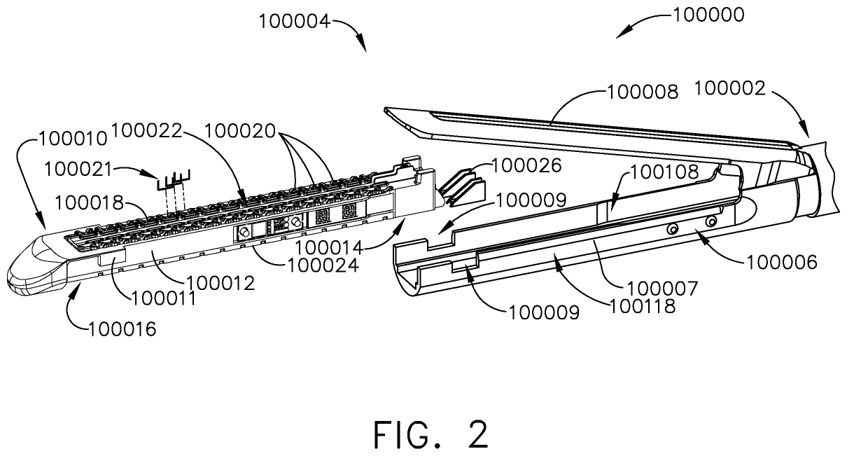

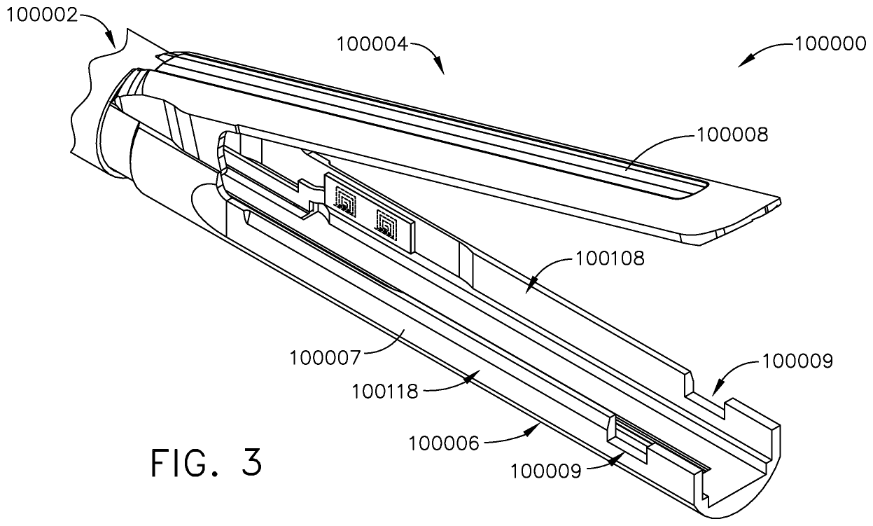

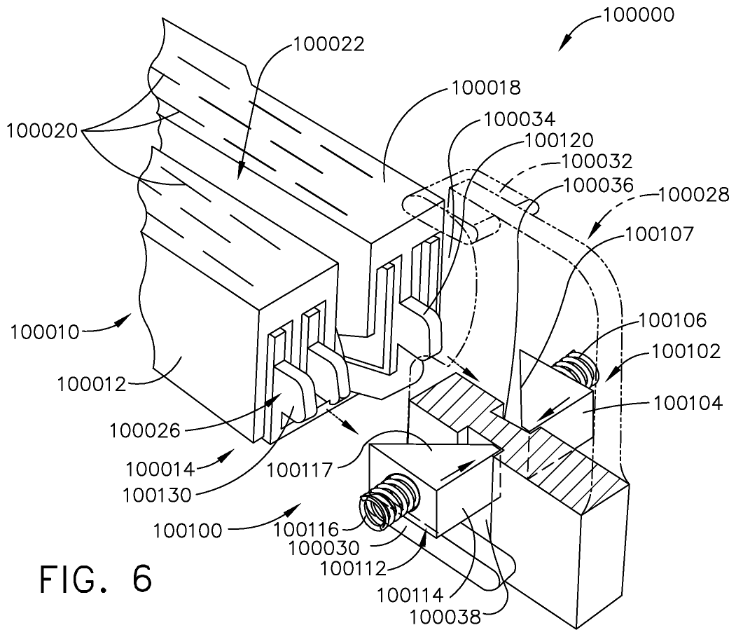

The staples are supported by staple drivers in the cartridge body 100012. Staples supported on staple drivers can be seen in U.S. Patent Application Publication No. 2021/0059672, entitled SURGICAL INSTRUMENT WITH WIRELESS COMMUNICATION BETWEEN A CONTROL UNIT OF A ROBOTIC SYSTEM AND REMOTE SENSOR, which is hereby incorporated by reference in its entirety herein. The drivers are movable between a first, or unfired position, and a second, or fired, position to eject the staples from the staple cavities 100020. The drivers are retained in the cartridge body 100012 by a retainer, or pan, 100024 which extends at least partially around the bottom of the cartridge body 100012 and includes resilient members configured to grip the cartridge body 100012 and hold the retainer 100024 to the cartridge body 100012. Alternatively, there may not be a retainer, or pan, attached to the cartridge body 100012. The drivers are movable between their unfired positions and their fired positions by a sled 100026 (see FIGS. 2 and 6). The sled 100026 is movable between a proximal position adjacent the proximal end 100014 and a distal position adjacent the distal end 100016. The sled 100026 comprises a plurality of ramped surfaces configured to slide under the drivers and lift the drivers, and the staples supported thereon, toward the anvil 100008. The staples may not be supported by staple drivers, but rather, the staples include integral drive surfaces that are directly engaged by the sled 100026 to lift the staples, examples of which are described in U.S. Patent Application Publication No. 2015/0173756, entitled SURGICAL CUTTING AND STAPLING METHODS, which is hereby incorporated by reference in its entirety herein.

Further to the above, the sled 100026 is moved distally by a firing actuator 100027 that comprises a firing driver 100028 and a firing bar extending proximally from the firing driver 100028. The firing driver 100028 is configured to contact the sled 100026 and push the sled 100026 toward the distal end 100016. The firing bar comprises a plurality of laminate strips; however, the firing bar may comprise a rod, for example. The longitudinal slot 100022 defined in the cartridge body 100012 is configured to receive the firing driver 100028 and the firing bar. The anvil 100008 also includes a slot defined therein that receives the firing driver 100028. The firing driver 100028 further comprises a first cam 100030 which engages the first jaw 100006 and a second cam 100032 which engages the second jaw 100008 in the slot of the anvil 100008. As the firing driver 100028 is advanced distally, the first cam 100030 and the second cam 100032 control the distance, or tissue gap, between the deck 100018 of the staple cartridge 100010 and the anvil 100008. The firing driver 100028 also comprises a knife 100034 configured to incise the tissue captured intermediate the staple cartridge 100010 and the anvil 100008. It is desirable for the knife 100034 to be positioned at least partially proximal to the ramped surfaces of the sled 100026 such that the staples are ejected ahead of the knife 100034. In addition, the firing driver 100028 comprises a first notch 100036 defined on a first lateral side thereof and a second notch 100038 defined on a second lateral side thereof.

The anvil 100008 can be moved from the open position to the closed position using a closure system that is controlled separately from the firing driver 100028. The firing driver is part of a firing actuator that is separate and distinctly operable from the closure system. The anvil 100008 can comprise a ramp on a proximal end thereof and the closure system comprises a closure tube that movable distally to engage the ramp and cam the anvil 100008 to the closed position. In the closed position, the first cam 100030 and the second cam 100032 of the firing driver 100028 translate distally and maintain the anvil 100008 in the closed position. To transition the anvil 100008 to the open position, the closure tube can be retracted proximally and springs positioned within the end effector 100004 can bias the anvil 100008 to the open position. The anvil 100008 can include a tab and the closure tube can define an aperture which engages the tab as the closure tube moves proximally, thereby positively transitioning the anvil 100008 to the open position. Exemplary closure systems and closure tubes are described in U.S. Patent Application Publication No. 2021/0059672, which was previously incorporated by reference in its entirety herein.

The anvil 100008 can be moved from the open position to the closed position using the firing driver 100028. As such, the anvil 100008 can include a ramp that extends proximally from the slot defined therein that is engaged by the firing driver 100028 during a first portion of the stroke of the firing driver 100028 to move the anvil 100008 to the closed position. The first portion of the stroke of the firing driver may be about 0.100″-0.250″ of the firing driver 100028 travel, for example. After the first portion of the stroke has been completed, the firing driver 100028 can be advanced distally through a second portion of the stroke to deploy staples from the staple cartridge 100010 and incise tissue captured by the end effector, as described above. Firing drivers that close the anvil and fire staples are described in U.S. Pat. No. 11,160,551. The firing driver 100028 may be driven distally by a drive beam extending therefrom. As such, the drive beam can comprise a plurality of laminated strips. The firing driver 100028 may be driven distally by an upper drive bar attached at an upper end of the firing driver 100028 and a lower drive bar attached to a lower end of the firing driver 100028, examples of which can be found in U.S. Patent Application Publication No. 2015/0173756, which is hereby incorporated by reference in its entirety herein.

After a staple cartridge 100010 has been fired, or at least partially fired, it can be removed from the elongate channel 100007 and then replaced with another replaceable staple cartridge, if desired. At such point, the surgical stapling system 100000 can be re-used to continue stapling and incising the patient tissue during a surgical procedure. However, a previously-fired staple cartridge can be accidentally loaded into the elongate channel 100007. If the firing driver 100028 were to be advanced distally within such a previously-fired staple cartridge, the surgical stapling system 100000 could cut the patient tissue without stapling it, absent more. The surgical stapling system 100000 could similarly cut the patient tissue without stapling it if the firing driver 100028, absent more, were advanced distally through a staple firing stroke without a staple cartridge positioned in the elongate channel 100007 at all. In addition, various surgical staple cartridges may have different arrays of and/or orientations of staples/fasteners therein. The sizes of the staples or fasteners, as well as the number of fasteners, for instance, may vary from cartridge type to cartridge type. To ensure that the staples are properly crimped or formed, it is often the case that surgical staple cartridges are paired with corresponding, compatible anvils that have a proper array of staple-forming pockets defined therein as well as proper cutting and firing components. Should a “non-compatible” cartridge be loaded into a surgical stapling device that has an anvil that is mismatched to the staple cartridge, the staples may not be properly formed during the firing process.

In view of the above, the surgical stapling system 100000 comprises a lockout system 100100 configured to prevent distal the firing driver 100028 from being moved distally unless a compatible and unspent staple cartridge is positioned in the first jaw 100006. The lockout system 100100 includes a first lockout 100102 that includes a first wedge 100104 and a first spring 100106 that biases the first wedge 100104 into the first notch 100036 of the firing driver 100028. The first spring 100106 is mounted to the elongate channel 100007 of the first jaw 100006 on a first lateral side 100108 thereof; however, the first spring 100106 can be mounted in any suitable location. The lockout system 100100 further includes a second lockout 100112 that includes a second wedge 100114 and a second spring 100116 that biases the second wedge 100114 into the second notch 100038 of the firing driver 100028. The second spring 100116 is mounted to the elongate channel 100007 of the first jaw 100006 on a second lateral side 100118 thereof; however, the second spring 100116 can be mounted in any suitable location. The first lockout 100102 and the second lockout 100112 can be mounted to the shaft 100002. Also, the springs 100106 and 100116 comprise coil springs, but can comprise any suitable biasing member.

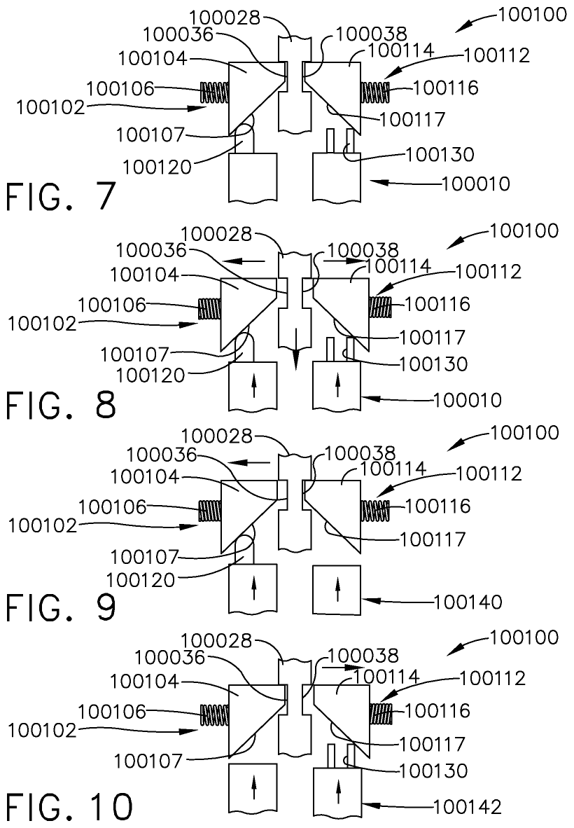

In use, the lockout system 100100 prevents advancement of the firing driver 100028 unless a compatible and unspent staple cartridge is seated in the first jaw 100006. The staple cartridge 100010 includes two discrete authentication keys—a first authentication key 100120 that is configured to transition the first lockout 100102 from a locked state (a state where the first wedge 100104 is positioned in the first notch 100036 of the firing driver 100028, see FIGS. 6 and 7) to an unlocked state (a state where the first wedge 100104 is displaced from the first notch 100036 (i.e. “defeated”) by the first authentication key 100120, see FIG. 8) and a second authentication key 100130 that is configured to transition the second lockout 100112 from a locked state (a state where the second wedge 100114 is positioned in the second notch 100038 of the firing driver 100028, see FIGS. 6 and 7) to an unlocked state (a state where the second wedge 100114 is displaced from the second notch 100038 by the second authentication key 100130 (i.e. “defeated”), see FIG. 8).

The lockouts 100102, 100112 are designed such that a single lockout by itself is able to prevent the firing driver 100028 from moving distally through the staple cartridge 100010. The spring and the wedge of each lockout 100102, 100112 are selected such that the lockout can resist the force being transmitted through the firing driver 100028 and stop the firing driver 100028 from being advanced further distally. As described in greater detail below, the lockout system 100100 ensures that staple cartridge positioned in the first jaw 100006 is both compatible with the stapling instrument 100000 and in an unspent condition in order to permit the firing driver 100028 to be moved through a firing stroke.

As shown in FIG. 6, the staple cartridge 100010 includes a first key 100120 that extends from the proximal end 100014 of the cartridge body 100012. As the staple cartridge 100010 is inserted into the first jaw 100006, the first key 100120 engages a first cam surface 100107 of the first wedge 100104. As the staple cartridge 100010 is seated in the elongate channel 100007, the first key 100120 cams the first wedge 100104 out of the first notch 100036, thus defeating the first lockout 100102 and placing the first lockout 100102 in the unlocked state.

The first lockout 100102 can be a compatible staple cartridge lockout as a compatible staple cartridge with a sufficient authentication key, such as the first key 100120 on the cartridge body 100012, transitions the first lockout 100102 to the unlocked position whereas a staple cartridge without the first key 100120, or a sufficient authentication key would not. The first key 100120, or a sufficient authentication key may be positioned on another portion of the staple cartridge, such as the retainer 100024, for example.

Further to the above, the staple cartridge 100010 includes a second key 100130 that extends from the proximal end of the sled 100026. Notably, as shown in FIG. 6, the sled 100026 is positioned in the proximal, unfired, position, indicating that the staple cartridge 100010 is an unspent staple cartridge (i.e., none of the staples from the staple cartridge have been deployed therefrom). As the staple cartridge 100010 is inserted into the first jaw 100006, the second key 100130 engages a second cam surface 100117 of the second wedge 100114. As the staple cartridge 100010 is seated in the elongate channel 100007, the second key 100130 cams the second wedge 100114 out of the second notch 100038, thus defeating the second lockout 100112 and placing the second lockout 100112 in the unlocked state.

The second lockout 100112 can be a spent staple cartridge lockout as a staple cartridge with a sled having a sufficient key in a sufficient location transitions the second lockout to the unlocked position whereas a staple cartridge without a sled having a sufficient key in a sufficient location would not. Should a clinician inadvertently positon a staple cartridge 100010 in the first jaw 100006 that is spent (i.e., all of the staples have been deployed from the staple cartridge), or partially spent (i.e., some, but not all, of the staples have been deployed from the staple cartridge), the second key 100130 would not be able to engage and cam the second wedge 100114 out of the second notch 100038 thereby preventing the firing driver 100028 from advancing distally through the staple cartridge even though the staple cartridge 100010 would unlock the first lockout 100102 with the first key 100120. The second key 100130 can extend a length from the sled 100026 such that the second key 100130 can only transition the second lockout 100112 to the unlocked position if the sled 100026 is in its proximal-most position or a position sufficiently close to the proximal-most position. The sled 100026 is in a sufficiently close position to the proximal-most position where none of the staples have been fired from the staple cartridge 100010 but the sled 100026 has been moved less than a threshold amount from the proximal-most position. In either such position, the sled 100026 is in a proximal unfired position and the second key 100130 would unlock the second lockout 100112 when the staple cartridge 100010 is seated in the first jaw 100006. If, however, the sled 100026 has already been moved distally past the threshold distance when the staple cartridge 100010 is seated in the first jaw 100006, the staple cartridge 100010 would be unable to transition the second lockout 100112 to the unlocked position and, as a result, the staple cartridge would need to be replaced with an unspent (and compatible) staple cartridge in order to fire the stapling instrument 100000. The second key 100130 can be positioned on another portion of the staple cartridge 100010 that is indicative of the firing status (unspent, spent, partially spent) of the staple cartridge 100010, such as a cover removably positioned over the deck 100018 of the staple cartridge 100010, for example.

FIGS. 7 and 8 illustrate the staple cartridge 100010 being inserted into the first jaw 100006 of the end effector 100004. Notably, the first authentication key 100120 engages the first cam surface 100107 of the first wedge 100104 to drive the first wedge 100104 out of the first notch 100036 and transition the first lockout 100102 to an unlocked state. Similarly, the second authentication key 100130 engages the second cam surface 100117 of the second wedge 100114 to drive the second wedge 100114 out of the second notch 100038 and transition the second lockout 100112 to an unlocked state. With both the first lockout 100102 and the second lockout 100112 in their respective unlocked states, the firing driver 100028 is free to translate through the longitudinal slot 100022 to deploy staples from the staple cartridge 100010 and incise tissue captured by the end effector 100004.

Referring now to FIG. 9, the staple cartridge 100010 has been replaced with a first replacement staple cartridge 100140 that includes a first key 100120 extending therefrom—but does not include a second key. An absent second key can be indicative of, for example, a staple cartridge that does not have a compatible sled even though the sled is in a proximal unspent position or, alternatively, a sled having a sufficient second key not being in an unfired position (i.e., a compatible sled being in at partially spent or spent position). Accordingly, when the first staple cartridge 100140 is removably positioned in the first jaw 100006, the first lockout 100102 is transitioned to the unlocked state (via the first key 100120) but the second lockout 100112 remains in the locked state. In such a state, the lockout system 100100 prevents distal motion of the firing driver 100028. More specifically, in such a state, the second wedge 100114 edges a trailing edge of the second notch 100038 as the firing driver 100028 is moved distally, preventing distal motion of the firing driver 100028.

Referring now to FIG. 10, the staple cartridge 100010 has been replaced with a second replacement staple cartridge 100142 that includes a second key 100130 extending therefrom—but not a first key. An absent first key from the second replacement staple cartridge 100142 can be indicative of the second replacement staple cartridge 100142 being an incompatible staple cartridge. Accordingly, when the second replacement staple cartridge 100142 is seated in the first jaw 100006, the second lockout 100112 is transitioned to the unlocked state (via the second key 100130) but the first lockout 100102 remains in the locked state. In such a state, the lockout system 100100 prevents distal motion of the firing driver 100028. More specifically, in such a state, the first wedge 100104 edges a trailing edge of the first notch 100036 as the firing driver 100028 is moved distally, preventing distal motion of the firing driver 100028.

Accordingly, the foregoing lockout system 100100 provides a “two-factor” authentication lockout which communicates to the clinician that, not only is the staple cartridge positioned in the first jaw 100006 a compatible staple cartridge, but also that the staple cartridge is ready-to-fire (i.e., unspent). This eliminates the possibility of a compatible staple cartridge that has already been spent, or at least partially spent, from being positioned in the first jaw 100006 of the end effector 100004. Furthermore, as outlined above, the lockout system 100100 includes two lockouts 100102, 100112 that each function to restrain the same drive system—the firing driver 100028. The firing driver 100028 is prevented from moving through the staple cartridge to both position the jaws of the end effector 100004 relative to one another and drive staples from the staple cartridge unless both lockouts 100102, 100112 are transitioned to their respective unlocked state via the authentication keys.

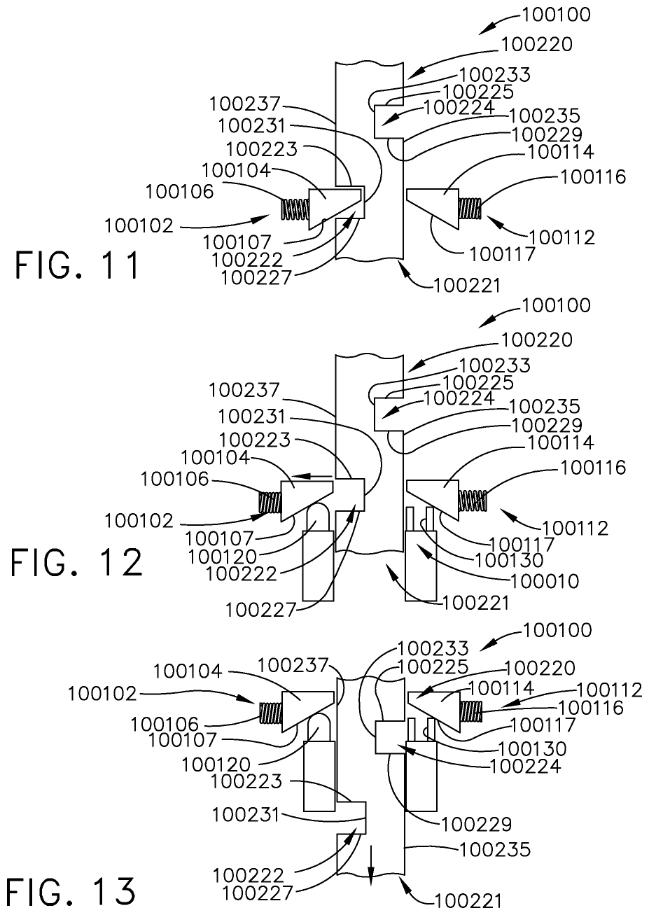

Referring now to FIG. 11, a firing driver 100220 for use with the lockout system 100100 is provided in accordance with the present disclosure. The firing driver 100220 is similar to the firing driver 100028 in many respects, with some differences noted below. The firing driver 100220 can be used in lieu of the firing driver 100028 in the firing actuator 100027.

Similar to the firing driver 100028, the firing driver 100220 comprises a first notch 100222 defined on a first lateral side thereof. The first notch 100222 comprises a first leading edge 100227 that is a first longitudinal distance from a distal end 100221 of the firing driver 100220, a first trailing edge 100223 that is a second longitudinal distance from the distal end 100221 of the firing driver 100220, and a first sidewall 100231 that extends between the first leading edge 100227 and the first trailing edge 100223. The distance between the first leading edge 100227 and the first trailing edge 100223 (i.e., the length of the first sidewall 100231) defines a width of the first notch 100222.

In addition, the firing driver 100220 comprises a second notch 100224 defined on a second lateral side thereof. The second notch 100224 comprises a second leading edge 100229 that is a third longitudinal distance from the distal end 100221 of the firing driver 100220, a second trailing edge 100225 that is a fourth longitudinal distance from the distal end 100221 of the firing driver 100220, and a second sidewall 100233 that extends between the second leading edge 100229 and the second trailing edge 100225. The distance between the second leading edge 100229 and the second trailing edge 100225 (i.e., the length of the second sidewall 100233) defines a width of the second notch 100254.

As shown in FIG. 11, the width of the second notch 100224 is the same as the width of the first notch 100222; however, the widths may be different, as will be described in more detail elsewhere herein. The second leading edge 100229 and the first leading edge 100227 can be different longitudinal distances from the distal end 100221 of the firing driver 100220; however, the leading edges 100227, 100229 may be the same distance from the distal end 100221 of the firing driver 100220. Furthermore, as shown in FIG. 11, the second trailing edge 100225 and the first trailing edge 100223 are different longitudinal distances from the distal end 100221 of the firing driver 100220; however, the longitudinal distances may be the same. For instance, the first notch 100036 can include a first trailing edge (similar to first trailing edge 100223) and the second notch 100038 can include a second trailing edge (similar to second trailing edge 100225) and both trailing edges are the same distance from the distal end of the firing driver 100028. As such, the trailing edges are able to co-operatively apply a resistance force to the firing driver if a user attempts to drive the firing driver though a firing stroke without a compatible unspent staple cartridge positioned in the end effector.

The firing driver 100220 can be usable with the end effector 100004 to both move the anvil 100008 from the open position (FIG. 5) to a closed position (FIG. 4) and then to fire the staples from the staple cartridge 100010. As referenced above, exemplary firing drivers that close the end effector and then fire staples from a staple cartridge are described in U.S. Pat. No. 11,160,551, entitled ARTICULATABLE SURGICAL STAPLING INSTRUMENTS, which is incorporated by reference in its entirety herein.

In operation, the firing driver 100220 is driven through a first motion and a second motion that is distal and subsequent to the first motion. During the first motion, the firing driver 100220 engages a ramp on the second jaw 100008 of the end effector 100004 to move the second jaw 100008 from the open position (FIG. 5) to the closed position (FIG. 4). The first motion of the firing driver 100220 can be about 0.100″-0.250″ of the firing driver 100220 travel, for example. During the second motion subsequent and distal to the first motion, the firing driver 100220 drives the sled 100026 through the staple cartridge 100010 to deploy the staples therefrom. The second motion of the firing driver 100220 can be the remainder of the firing driver 100220 travel after the first motion.

As shown in FIG. 11 and described above, the firing driver 100220 comprises two longitudinally offset notches 100222, 100224. In FIG. 11, the firing driver 100220 is in a proximal position. In the proximal position, the first notch 100222 receives the first wedge 100104, which corresponds to the first lockout 100102 being in the locked state. In addition, in the proximal position, the second wedge 100114 engages a sidewall 100235 of the firing driver 100220, maintaining the second lockout 100112 in the unlocked state. As described elsewhere herein, each of the lockouts 100102, 100112 are designed such that the firing driver 100220 is unable to proceed distally so long as one of the lockouts is in its locked state. Accordingly, as shown in FIG. 11, the firing driver 100220 is unable to proceed distally as the first lockout 100102 is in its locked state. In particular, the first wedge 100104 of the first lockout 100102 engages the first trailing edge 100223 of the first notch 100222, preventing distal motion of the firing driver 100220.

When a compatible and unspent staple cartridge (such as staple cartridge 100010) is inserted into the first jaw 100006 (as shown in FIG. 12), the first authentication key 100120 engages and cams the first wedge 100104 out of the first notch 100222 transitioning the first lockout 100102 to the unlocked state. In addition, the second authentication key 100130 engages the second wedge 100114 to maintain the second lockout 100112 in the unlocked state. Accordingly, the firing driver 100220 is able to proceed distally to move the anvil 100008 to the closed position and fire staples from the staple cartridge 100010, as shown in FIG. 13.

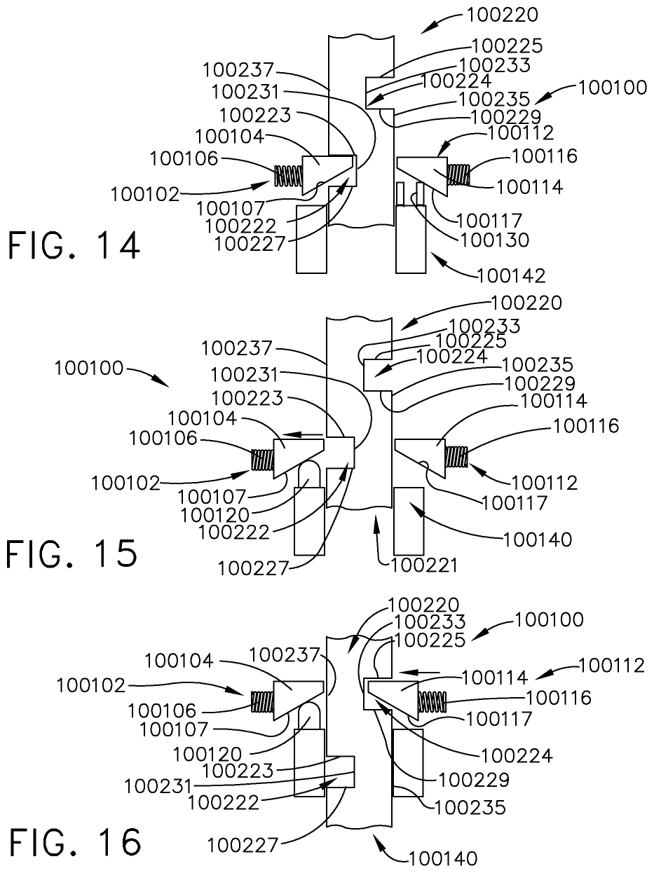

Referring now to FIG. 14, when an incompatible staple cartridge, such as staple cartridge 100142, is inserted into the first jaw 100006, the second authentication key 100130 engages the second wedge 100114 to maintain the second lockout 100112 in the locked state. However, notably, the first staple cartridge 100142 does not include a first authentication key 100120, and therefore, the first lockout 100102 remains in the locked position, preventing distal motion of the firing driver 100220. In particular, the first wedge 100104 of the first lockout 100102 engages the first trailing edge 100223 of the notch 100222 preventing distal motion of the firing driver 100220.

Referring now to FIGS. 15 and 16, when an incompatible staple cartridge, such as staple cartridge 100140, is inserted into the first jaw 100006, the first authentication key 100120 engages and cams the first wedge 100104 out of the first notch 100222, transitioning the first lockout 100102 to the unlocked state. However, notably, the staple cartridge 100140 does not include a second authentication key 100130 to engage and maintain the second lockout 100112 in the unlocked position. However, as referenced above, the second wedge 100114 is engaged with the sidewall 100235 of the firing driver 100220, which holds the second lockout 100112 in the unlocked state. Accordingly, the firing driver 100220 is able to translate distally through a first portion of its firing stroke.

The first stroke portion can comprise the first motion, described above, in which the firing driver 100220 engages a ramp on the second jaw 100008 of the end effector 100004 to move the anvil 100008 from the open position (FIG. 5) to the closed position (FIG. 4). The first stroke portion can comprise about 0.100″-0.250″ of the firing driver 100220 travel, for example. The first stroke portion can be defined as the distance until the second wedge 100114 engages the second trailing edge 100225 of the second notch 100224, described in more detail below. During the first stroke portion, the second wedge 100114 of the second lockout 100112 rides along the sidewall 100235 of the firing driver 100220 and the first wedge 100104 of the first lockout 100102 moves along another sidewall 100237 of the firing driver 100220. During the first motion, the wedge 10114 applies a frictional force to the sidewall 100235 of the firing driver 100220; however, the frictional force is insufficient to prevent distal movement of the firing driver 100220.

Once the firing driver 100220 has translated distally through the first stroke portion, s shown in FIG. 16, the second wedge 100114 becomes aligned with the second notch 100224. As the staple cartridge 100140 does not include a second authentication key 100130 to engage and maintain the second lockout 100112 in the unlocked state, the second spring 100116 biases the second wedge 100114 into the second notch 100224 preventing further distal movement of the firing driver 100220. In particular, the second wedge 100114 of the second lockout 100112 engages the second trailing edge 100225 of the second notch 100224 preventing distal motion of the firing driver 100220.

Accordingly, utilizing the firing driver 100220 with the lockout system 100100 enables to clinician to transition the end effector 100004 between an open state (FIG. 5) and a closed state (FIG. 4) to grasp tissue, for example, when a staple cartridge with a first authentication key 100120 is positioned in the end effector. Such a staple cartridge could comprise a spent staple cartridge 100010, for example. However, owing to the absence of a sufficient second authentication key, the firing driver 100220 is prevented from further advancing through the staple cartridge. The firing driver 100220 allows for a staple cartridge that includes a first authentication key 100120 to maintain some functionality (i.e., allowing for opening and closing of the end effector 100004), but preventing the firing driver 100220 from proceeding through a portion of its stroke that could result in damage to the patient tissue (moving the knife through the staple cartridge that may not include staples therein).

Furthermore, the inclusion of the second cam surface 100117 on the second wedge 100114 enables a clinician to move the firing driver 100220 proximally after the second lockout 100112 has transitioned to the locked state (i.e., when the second wedge 100114 is positioned in the second notch 100224). In particular, as the firing driver 100220 moves proximally, the second cam surface 100117 engages the second leading edge 100229 of the second notch 100224, which cams the second wedge 100114 out of the second notch 100224 and reengages the second wedge 100114 with the sidewall 100235.

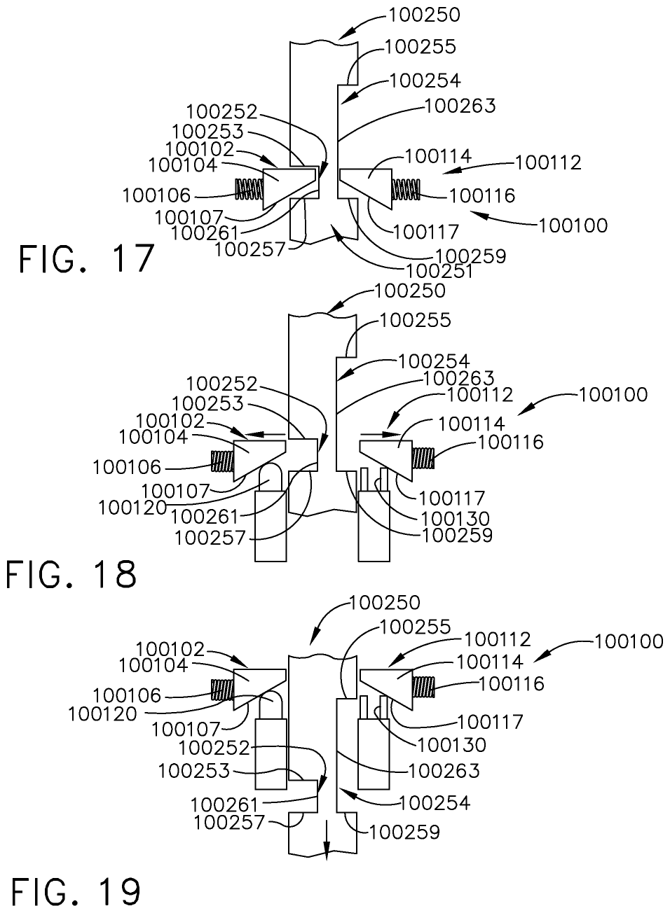

Referring now to FIG. 17, a firing driver 100250 for use with the lockout system 100100 is provided in accordance with the present disclosure. The firing driver 100250 is similar to the firing driver 100028 and the firing driver 100220 in many respects, with some differences noted below. The firing driver 100250 can be used in lieu of the firing driver 100028 or firing driver 100250 in the firing actuator 100027.

Similar to the firing driver 100028 and the firing driver 100220, the firing driver 100220 comprises a first notch 100252 defined on a first lateral side thereof. The first notch 100252 comprises a first leading edge 100257 that is a first longitudinal distance from the distal end 100251 of the firing driver 100250, a first trailing edge 100253 that is a second longitudinal distance from the distal end 100251 of the firing driver 100250, and a first sidewall 100261 that extends between the first leading edge 100257 and the first trailing edge 100253. The distance between the first leading edge 100257 and the first trailing edge 100253 (i.e., the length of the first sidewall 100261) defines a width of the first notch 100252.

In addition, the firing driver 100220 comprises a second notch 100254 defined on a second lateral side thereof. The second notch 100254 comprises a second leading edge 100259 that is a third longitudinal distance from the distal end 100251 of the firing driver 100250, a second trailing edge 100255 that is a fourth longitudinal distance from the distal end 100251 of the firing driver 100250, and a second sidewall 100263 that extends between the second leading edge 100259 and the second trailing edge 100255. The distance between the second leading edge 100259 and the second trailing edge 100255 (i.e., the length of the second sidewall 100263) defines a width of the second notch 100254.

As shown in FIG. 17, the width of the second notch 100254 is different than the width of the first notch 100252. In particular, the width of the second notch 100254 is greater than the width of the first notch 100252. The second leading edge 100259 and the first leading edge 100257 can be the same longitudinal distance from the distal end 100251 of the firing driver 100250; however, the leading edges 100257, 100259 can be different distances from the distal end 100251 of the firing driver 100250. The second trailing edge 100255 can be a greater longitudinal distance from the distal end 100251 of the firing driver 100250 than the longitudinal distance of the first trailing edge 100253 to the distal end 100251 of the firing driver 100250.

The firing driver 100250 can be usable with the end effector 100004 to both move the anvil 100008 from the open position (FIG. 5) to a closed position (FIG. 4) and then to fire the staples from the staple cartridge 100010. As referenced above, firing drivers that close the end effector and then fire staples from a staple cartridge are described in U.S. Pat. No. 11,160,551, which is incorporated by reference in its entirety herein.

In operation, the firing driver 100250 is driven through a first motion and a second motion that is distal and subsequent to the first motion. During the first motion, the firing driver 100250 engages a ramp on the second jaw 100008 of the end effector 100004 to move the second jaw 100008 from the open position (FIG. 5) to the closed position (FIG. 4). The first motion of the firing driver 100250 can be about 0.100″-0.250″ of the firing driver 100220 travel, for example. During the second motion subsequent and distal to the first motion, the firing driver 100250 drives the sled 100026 through the staple cartridge 100010 to deploy the staples therefrom. The second motion of the firing driver 100250 can be the remainder of the firing driver 100250 travel after the first motion.

As described above, the firing driver 100250 comprises two notches, i.e., notches 100252 and 100254, that comprise different widths. As shown in FIG. 17, the firing driver 100250 is in a proximal position. In the proximal position, the first notch 100252 receives the first wedge 100104, which corresponds to the first lockout 100102 being in the locked state. In addition, the second notch 100254 receives the second wedge 100114, which corresponds to the second lockout 100112 being in the locked state. The firing driver 100250 can be capable of translating with one of the lockouts in a locked state, as described in more detail below.

When a compatible and unspent staple cartridge (such as staple cartridge 100010) is inserted into the first jaw 100006 (as shown in FIG. 18), the first authentication key 100120 engages and cams the first wedge 100104 out of the first notch 100252, transitioning the first lockout 100102 to the unlocked state. In addition, the second authentication key 100130 engages and cams the second wedge 100114 out of the second notch 100254, transitioning the second lockout 100112 to the unlocked state. Accordingly, the firing driver 100250 is able to proceed distally to move the anvil 100008 to the closed position and fire staples from the staple cartridge 100010, as shown in FIG. 19.

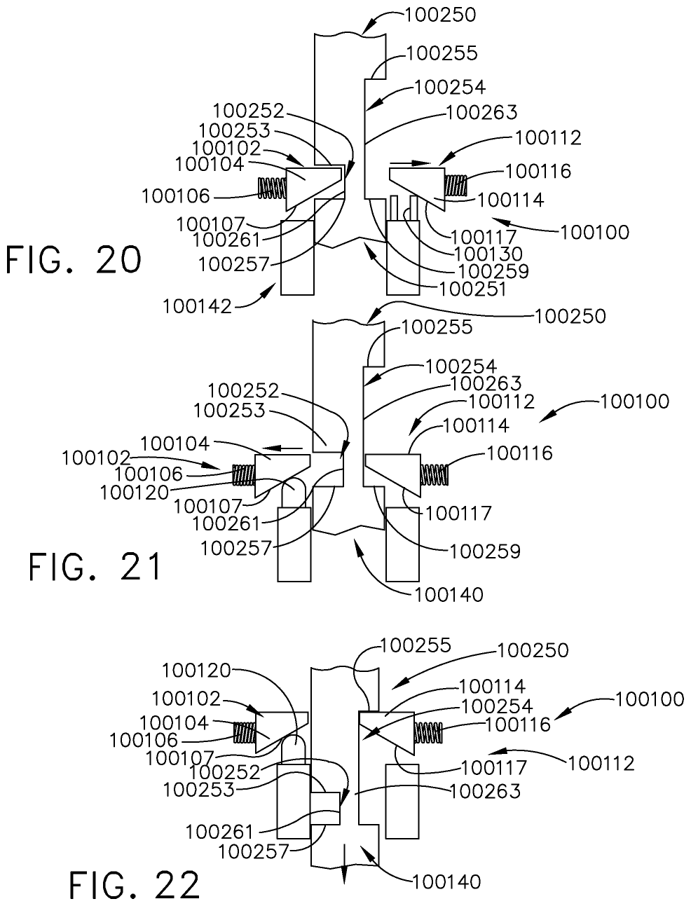

Referring now to FIG. 20, when an incompatible staple cartridge such as staple cartridge 100142 is inserted into the first jaw 100006, the second authentication key 100130 engages and cams the second wedge 100114 out of the second notch 100254 transitioning the second lockout 100112 to the unlocked state. However, notably, the first staple cartridge 100142 does not include a first authentication key 100120 and, therefore, the first lockout 100102 remains in the locked position preventing distal motion of the firing driver 100250. In particular, the first wedge 100104 of the first lockout 100102 engages the first trailing edge 100253 of the first notch 100252 preventing distal motion of the firing driver 100250.

Referring now to FIGS. 21 and 22, when an incompatible staple cartridge such as staple cartridge 100140 is inserted into the first jaw 100006, the first authentication key 100120 engages and cams the first wedge 100104 out of the first notch 100252 transitioning the first lockout 100102 to the unlocked state. However, notably, the staple cartridge 100140 does not include a second authentication key 100130 to engage and cam the second lockout 100112 to the unlocked position. As seen in FIG. 21, the second trailing edge 100255 of the second notch 100254 is positioned proximally with respect to the second wedge 100114, and therefore, the second trailing edge 100255 cannot prevent motion of the firing driver 100250. Accordingly, the firing driver 100250 is able to translate distally through a first portion of its stroke even though the second lockout 100112 is still in a locked condition. During the first portion of the stroke, the wedge 10114 applies a friction force to the second sidewall 100263 of the firing driver 100250; however, the friction force is insufficient to prevent distal movement of the firing driver 100250.

Further to the above, the first stroke portion comprises the first motion, described above, in which the firing driver 100250 engages a ramp on the second jaw 100008 of the end effector 100004 to move the anvil 100008 from the open position (FIG. 5) to the closed position (FIG. 4). The first stroke portion can comprise about 0.100″-0.250″ of the firing driver 100250 travel, for example. The first stroke portion can be defined as the distance between the second wedge 100114 and the second trailing edge 100255, and therefore, the distance that the firing driver 100250 can travel until the second wedge 100114 blocks the second trailing edge 100255 to prevent further displacement of the firing driver 100250, as shown in FIG. 22.

Utilizing the firing driver 100250 with the lockout system 100100 enables to clinician to transition the end effector 100004 between an open state (FIG. 5) and a closed state (FIG. 4) to grasp tissue, for example, when a staple cartridge with a first authentication key is positioned in the end effector. However, absent a sufficient second authentication key, the firing driver 100250 is prevented from further advancing through the staple cartridge. The firing driver 100250 allows for a staple cartridge that includes a first authentication key to maintain some functionality (i.e., allowing for opening and closing of the end effector 100004), but preventing the firing driver 100250 from proceeding through a portion of its stroke that could result in damage to the patient tissue (moving the knife through the staple cartridge that may not include staples therein).

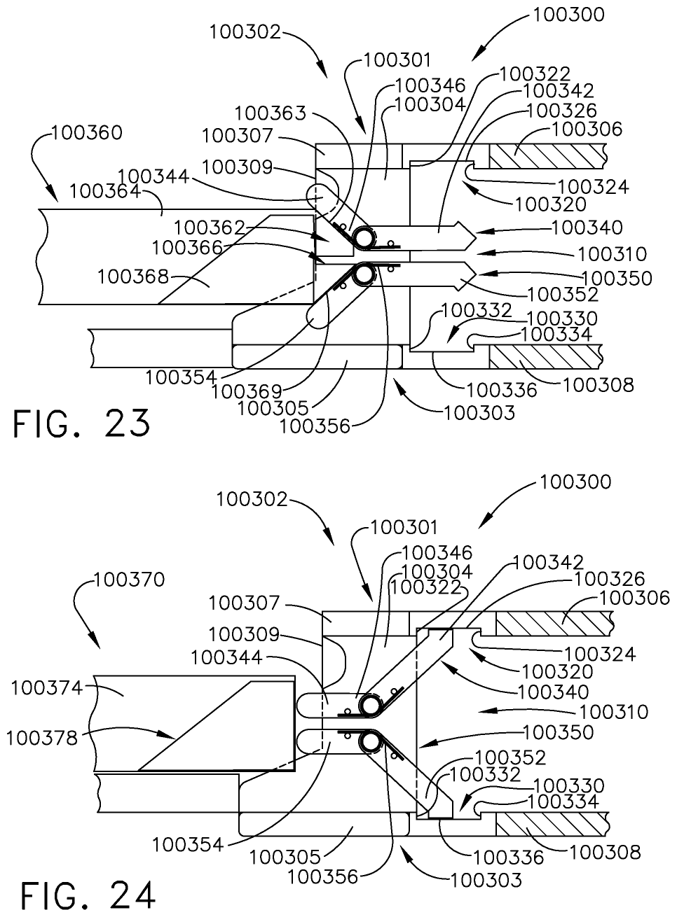

Referring now to FIG. 23, a surgical stapling system 100300 is provided that comprises a firing actuator 100302 and a lockout system 100310. The firing actuator 100302 and the lockout system 100310 can be used with the end effector 100004. The firing actuator 100302 and the lockout system 100310 can be used in lieu of the firing driver 100028 and the lockout system 100100 in the surgical stapling system 100000.

The firing actuator 100302 comprises a firing driver 100304, a first push rod 100306 attached at an upper end 100301 of the firing driver 100304, and a second push rod 100308 attached at a lower end 100303 of the firing driver 100304. The first push rod 100306 and the second push rod 100308 can be driven using any suitable drive system, such as a motor drive system, to drive the firing driver 100304 from a proximal position, shown in FIG. 23, to a distal position. An exemplary motor drive system is described in U.S. Patent Application Publication No. 2015/0173756, which is hereby incorporated by reference in its entirety herein. The firing driver 100304 further comprises a first cam 100305 configured to engage a first jaw of an end effector, a second cam 100307 configured to engage a second jaw of the end effector, and a knife 100309 configured to cut tissue after the end effector has been closed. As the firing driver 100304 is advanced distally, the first cam 100305 and the second cam 100307 control the distance, or tissue gap, between the deck of a staple cartridge and an anvil. Driving the firing driver 100304 toward the distal position causes staples to be deployed from a staple cartridge, as described elsewhere herein. Driving the firing driver 100304 may distally rotate the anvil toward a closed position (first stroke portion) and then cause staples to be deployed from a staple cartridge (see stroke portion) when the firing driver 100304.

The first push rod 100306 comprises a first notch 100320 defined therein. The first notch 100320 comprises a first leading edge 100322, a first trailing edge 100324, and a first sidewall 100326 extending from the first leading edge 100322 to the first trailing edge 100324. Similarly, the second push rod 100308 comprises a second notch 100330 defined therein. The second notch 100330 comprises a second leading edge 100332, a second trailing edge 100334, and a second sidewall 100336 extending from the second leading edge 100332 to the second trailing edge 100334.

The lockout system 100310 comprises a first lockout 100340 that comprises a first lockout arm 100342, a first cam surface 100344, and a first spring 100346. The first lockout 100340 is pivotable between an unlocked state (a state where the first lockout arm 100342 is rotated within first notch 100320, see FIG. 24) and an unlocked state (a state where the first lockout arm 100342 is rotated out of the first notch 100320, see FIG. 23). The first spring 100346 rotatably biases the first lockout 100340 toward the locked state. The first lockout 100340 is pivotably coupled to an elongate channel of an end effector, such as elongate channel 100007 of end effector 100004, for example, but can be pivotably coupled at any suitable location in the stapling instrument, such as in the shaft, for example.

The lockout system 100310 further comprises a second lockout 100350 that comprises a second lockout arm 100352, a second cam surface 100354, and a second spring 100356. The second lockout 100350 is pivotable between an unlocked state (a state where the second lockout arm 100352 is rotated within the second notch 100330, see FIG. 24) and an unlocked state (a state where the second lockout arm 100352 is rotated out of the second notch 100330, see FIG. 23). The second spring 100356 rotatably biases the second lockout 100350 toward the locked state. The second lockout 100350 is pivotably coupled to an elongate channel of an end effector, such as elongate channel 100007 of end effector 100004, for example, but can be pivotably coupled at any suitable location in the stapling instrument, such as in the shaft, for example. The first lockout arm 100342 and the second lockout arm 100352 may be pivotably coupled to the same lateral side of the elongate channel. Alternatively, the first lockout arm 100342 and the second lockout arm 100352 may be pivotably coupled to different lateral sides of the elongate channel.

In use, the lockout system 100310 prevents advancement of the firing driver 100304 unless a compatible and unspent staple cartridge is positioned in the first jaw of the end effector. Similar to the above, the staple cartridge includes two discrete and separately functioning authentication keys where a first authentication key is configured to transition the first lockout 100340 from the locked state to the unlocked state and a second authentication key is configured to transition the second lockout 100350 from the locked state to the unlocked state, as described in greater detail below.

The first lockout 100340 and the second lockout 100350 are designed such that either one of the lockouts by itself is able to prevent the firing driver 100304 from moving distally through the staple cartridge. The lockout arms and the springs are configured such that the lockouts are able to separately block one of the drive rods of the firing driver 100304. A staple cartridge, such as staple cartridge 100360, for example, includes a first key 100362 that extends from the proximal end of its cartridge body 100364 and includes a first camming surface 100363. As the staple cartridge 100360 is seated in the elongate channel, the first camming surface 100363 engages the first cam surface 100344 to rotate the first lockout 100340 to the unlocked state. With the first lockout 100340 in the unlocked state, the first push rod 100306 is permitted to drive the firing driver 100304 through the staple cartridge 100360 (should the second lockout 100350 also being in the unlocked state, as described in more detail below).

Further to the above, the staple cartridge 100360 includes a second key 100366 that extends from the proximal end of a sled 100368 and includes a second camming surface 100369. Notably, as shown in FIG. 23, the sled 100368 is positioned in a proximal, unfired, position, indicating that the staple cartridge 100360 is an unspent staple cartridge (i.e., none of the staples from the staple cartridge have been deployed therefrom). As the staple cartridge 100360 is removably positioned in the first jaw, the second key 100366 engages the second lockout 100350. As the staple cartridge 100360 is moved proximally to seat the staple cartridge 100360 in the elongate channel, the second camming surface 100369 engages the second cam surface 100354 to rotate the second lockout 100350 to the unlocked state. With the second lockout 100350 in the unlocked state, the second push rod 100308 is permitted to drive the firing driver 100304 through the staple cartridge 100360 (should the first lockout 100340 also being in the unlocked state, as described above).

Further to the above, the first lockout 100340 comprises a compatibility lockout. The second lockout 100350 comprises a spent cartridge lockout but also serves as a compatibility lockout. In order to unlock the second lockout 100350, two conditions regarding the sled of a staple cartridge must be met—the sled must be in a proximal unfired position and the sled must have a sufficient authentication key. In use, should a clinician attempt to drive the firing driver 100304 distally with one or both of the lockouts in their respectively locked position, the lockout arm thereof will engage a corresponding trailing edge of the notch to halt distal advancement of the firing driver 100304. Similar to the above, the first notch 100320 and the second notch 100330 can include a width (distance from leading edge to trailing edge) that enables the firing driver 100304 to move through a first stroke portion to move the anvil to the closed position prior to the lockout arms engaging their respective trailing edges to halt the distal translation of the firing driver 100304. Accordingly, the lockout system 100310 would still enable a clinician to transition the end effector between the open position and the closed position even if the installed staple cartridge is not authentic and/or is in a spent condition but not enable the firing driver 100304 to pass through the staple cartridge. Referring now to FIG. 24, the staple cartridge 100360 has been replaced with a first replacement staple cartridge 100370 that includes neither a first key on its cartridge body 100374 nor a second key on its sled 100378. Accordingly, when the first replacement staple cartridge 100370 is seated in the first jaw, both the first lockout 100340 and the second lockout 100350 remain in their respective locked positions but, as described above, the firing driver 100304 can still be moved distally to close the end effector. Alternatively, distal motion of the firing driver 100304 can be prevented with one or both of the lockouts in their respective locked position and the end effector cannot be closed by the firing driver 100304 unless both lockouts have been defeated.

Many of the surgical instrument systems described herein are motivated by an electric motor; however, the surgical instrument systems described herein can be motivated in any suitable manner. In accordance with the present disclosure, the surgical instrument systems described herein can be motivated by a manually-operated trigger, for example. The motors disclosed herein may comprise a portion or portions of a robotically controlled system. Moreover, any of the end effectors and/or tool assemblies disclosed herein can be utilized with a robotic surgical instrument system. U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535, for example, discloses several examples of a robotic surgical instrument system in greater detail.

Although various devices have been described herein in connection with certain embodiments, modifications and variations to those embodiments may be implemented. Particular features, structures, or characteristics may be combined in any suitable manner in one or more embodiments. Thus, the particular features, structures, or characteristics illustrated or described in connection with one embodiment may be combined in whole or in part, with the features, structures or characteristics of one ore more other embodiments without limitation. Also, where materials are disclosed for certain components, other materials may be used. Furthermore, according to various embodiments, a single component may be replaced by multiple components, and multiple components may be replaced by a single component, to perform a given function or functions. The foregoing description and following claims are intended to cover all such modification and variations.

Various aspects of the subject matter described herein are set out in the following numbered examples:

Example 1—A surgical stapling system (100000, 100300) comprising an end effector (100004), a firing actuator (100027, 100302) movable from a proximal position toward a distal position during a firing stroke, a first lockout (100102, 100340), and a second lockout (100112, 100350). The end effector comprises a first jaw (100006) and a second jaw (100008) pivotable relative to the first jaw between an open position and a closed position. The firing stroke comprises a first stroke portion and a second stroke portion distal to the first stroke portion. The firing actuator comprises a first edge (100223, 100253, 100324) and a second edge (100233, 100255, 100334). The first lockout is configurable in a first locked state in which the first lockout engages the first edge during the firing stroke and a first unlocked state in which the first edge bypasses the first lockout during the firing stroke. A staple cartridge (100010, 100360) comprising a first key (100120, 100362) is configured to place the first lockout in the first unlocked state based on the staple cartridge being positioned in the end effector. The second lockout is configurable in a second locked state in which the second lockout engages the second edge during the firing stroke and a second unlocked state in which the second edge bypasses the second lockout during the firing stroke. A staple cartridge (100360) comprising a second key (100130, 100366) is configured to place the second lockout in the second unlocked state based on the staple cartridge being positioned in the end effector.

Example 2—The surgical stapling system of Example 1, wherein the firing actuator is prevented from moving through the firing stroke absent a staple cartridge comprising the first key and the second key being positioned in the end effector.

Example 3—The surgical stapling system of Example 1, wherein the firing actuator is permitted to move through the first stroke portion but prevented from moving through the second stroke portion based on a staple cartridge being positioned in the end effector that comprises the first key but does not comprise the second key.

Example 4—The surgical stapling system of Example 3, wherein the firing actuator comprises a sidewall (100235), wherein the second lockout is configured to engage the sidewall to maintain the second lockout in the second unlocked state during the first stroke portion.

Example 5—The surgical stapling system of Example 3, wherein the firing actuator is permitted to move through the first stroke portion with the second lockout being in the second locked state.

Example 6—The surgical stapling system of Example 1 wherein the first edge is a first length from a distal end thereof, the second edge is a second length from the distal end thereof, and the first length is the same as the second length.

Example 7—The surgical stapling system of Example 1, wherein the first edge is a first length from a distal end thereof, the second edge is a second length from the distal end thereof, and the second length is different than the first length.

Example 8—The surgical stapling system of Example 1, wherein the firing actuator defines a first notch (100036, 100222, 100252, 100320) and a second notch, the first notch comprises the first edge and a first sidewall (100231, 100261, 100326) extending from the first edge, the second notch comprises the second edge and a second sidewall (100233, 100263, 100336) extending from the second edge, the first sidewall comprises a first width, the second sidewall comprises a second width, and the second width is the same as the first width.

Example 9—The surgical stapling system of Example 1, wherein the firing actuator defines a first notch (100036, 100222, 100252, 100320) and a second notch, the first notch comprises the first edge and a first sidewall (100231, 100261, 100326) extending from the first edge, the second notch comprises the second edge and a second sidewall (100233, 100263, 100336) extending from the second edge, the first sidewall comprises a first width, the second sidewall comprises a second width, and the second width is different than the first width.

Example 10—The surgical stapling system of any one of Examples 1-9, wherein the firing actuator comprises a firing driver (100028, 100220, 100250) and a firing bar extending from the firing driver, and wherein the firing driver comprises the first edge and the second edge.

Example 11—The surgical stapling system of any one of Examples 1-9, wherein the firing actuator comprises a firing driver (100304), a first push rod (100306) operably coupled to an upper end (100301) of the firing driver, wherein the first push rod comprises the first edge, and a second push rod (100308) operably coupled to a lower end (100303) of the firing driver, wherein the second push rod comprises the second edge.

Example 12—The surgical stapling system of Example 1, wherein the first lockout comprises a first wedge (100104) and a first spring (100106) configured to bias the first wedge toward the first edge and the second lockout comprises a second wedge (100114) and a second spring (100116) configured to bias the second wedge toward the second edge.

Example 13—The surgical stapling system of Example 12, wherein the first wedge comprises a first cam surface (100107). The first key is configured to engage the first cam surface to move the first wedge away from the first edge. The second wedge comprises a second cam surface (100117). The second key is configured to engage the second cam surface to move the second wedge away from the second edge.

Example 14—The surgical stapling system of Example 1, wherein the first lockout comprises a first lockout arm (100342) pivotably coupled to the first jaw and a first spring (100346) configured to bias the first lockout arm toward the first edge. The second lockout comprises a second lockout arm (100352) pivotably coupled to the first jaw and a second spring (100356) configured to bias the second lockout arm toward the second edge.

Example 15—The surgical stapling system of Example 14, wherein the first lockout arm comprises a first cam surface (100344) and the second lockout arm comprises a second cam surface (100354). The first key is configured to engage the first cam surface to rotate the first lockout arm away from the first edge. The second key is configured to engage the second cam surface to rotate the second lockout arm away from the second edge.

Example 16—A surgical stapling system comprising an end effector, a firing actuator movable from a proximal position toward a distal position during a firing stroke, a first lockout, and a second lockout. The end effector comprises a first jaw and a second jaw pivotable relative to the first jaw between an open position and a closed position. The firing stroke comprises a first stroke portion and a second stroke portion distal to the first stroke portion. The firing actuator comprises a first edge and a second edge. The first lockout is configurable in a first locked state in which the first lockout engages the first edge during the firing stroke and a first unlocked state in which the first edge bypasses the first lockout during the firing stroke. A staple cartridge comprising a first key is configured to place the first lockout in the first unlocked state based on the staple cartridge being positioned in the end effector. The second lockout is configurable in a second locked state in which the second lockout engages the second edge during the firing stroke and a second unlocked state in which the second edge bypasses the second lockout during the firing stroke. A staple cartridge comprising a second key is configured to place the second lockout in the second unlocked state based on the staple cartridge being positioned in the end effector.

Example 17—The surgical stapling system of Example 16, wherein the firing actuator is prevented from moving through the firing stroke absent a staple cartridge comprising the first key and the second key being positioned in the end effector.

Example 18—The surgical stapling system of Example 16, wherein the firing actuator is permitted to move through the first stroke portion but prevented from moving through the second stroke portion based on a staple cartridge being positioned in the end effector that comprises the first key but does not comprise the second key.

Example 19—The surgical stapling system of Example 18, wherein the firing actuator comprises a sidewall, wherein the second lockout is configured to engage the sidewall to maintain the second lockout in the second unlocked state during the first stroke portion.

Example 20—The surgical stapling system of Example 18, wherein the firing actuator is permitted to move through the first stroke portion with the second lockout being in the second locked state.

Example 21—The surgical stapling system of Example 16 wherein the first edge is a first length from a distal end thereof, the second edge is a second length from the distal end thereof, and the first length is the same as the second length.

Example 22—The surgical stapling system of Example 16, wherein the first edge is a first length from a distal end thereof, the second edge is a second length from the distal end thereof, and the second length is different than the first length.

Example 23—The surgical stapling system of Example 16, wherein the firing actuator defines a first notch and a second notch, the first notch comprises the first edge and a first sidewall extending from the first edge, the second notch comprises the second edge and a second sidewall extending from the second edge, the first sidewall comprises a first width, the second sidewall comprises a second width, and the second width is the same as the first width.

Example 24—The surgical stapling system of Example 16, wherein the firing actuator defines a first notch and a second notch, the first notch comprises the first edge and a first sidewall extending from the first edge, the second notch comprises the second edge and a second sidewall extending from the second edge, the first sidewall comprises a first width, the second sidewall comprises a second width, and the second width is different than the first width.

Example 25—The surgical stapling system of Example 16, wherein the firing actuator comprises a firing driver and a firing bar extending from the firing driver, and wherein the firing driver comprises the first edge and the second edge.

Example 26—The surgical stapling system of Example 16, wherein the firing actuator comprises a firing driver, a first push rod operably coupled to an upper end of the firing driver, wherein the first push rod comprises the first edge, and a second push rod operably coupled to a lower end of the firing driver, wherein the second push rod comprises the second edge.

Example 27—The surgical stapling system of Example 16, wherein the first lockout comprises a first wedge and a first spring configured to bias the first wedge toward the first edge and the second lockout comprises a second wedge and a second spring configured to bias the second wedge toward the second edge.

Example 28—The surgical stapling system of Example 27, wherein the first wedge comprises a first cam surface. The first key is configured to engage the first cam surface to move the first wedge away from the first edge. The second wedge comprises a second cam surface. The second key is configured to engage the second cam surface to move the second wedge away from the second edge.

Example 29—The surgical stapling system of Example 16, wherein the first lockout comprises a first lockout arm pivotably coupled to the first jaw and a first spring configured to bias the first lockout arm toward the first edge. The second lockout comprises a second lockout arm pivotably coupled to the first jaw and a second spring configured to bias the second lockout arm toward the second edge.

Example 30—The surgical stapling system of Example 29, wherein the first lockout arm comprises a first cam surface and the second lockout arm comprises a second cam surface. The first key is configured to engage the first cam surface to rotate the first lockout arm away from the first edge. The second key is configured to engage the second cam surface to rotate the second lockout arm away from the second edge.

Example 31—A surgical stapling system comprising an end effector, a firing driver movable from a proximal position toward a distal position during a firing stroke, a first lockout, a second lockout, and a staple cartridge. The end effector comprises an elongate channel and an anvil pivotable relative to the elongate channel between an open position and a closed position. The firing driver comprises a first shoulder and a second shoulder. The first lockout is configurable between a first locked state in which the first lockout engages the first shoulder during the firing stroke and a first unlocked state in which the first shoulder bypasses the first lockout during the firing stroke. The second lockout is configurable between a second locked state in which the second lockout engages the second shoulder during the firing stroke and a second unlocked state in which the second shoulder bypasses the second lockout during the firing stroke. The staple cartridge is configured to be removably positioned in the elongate channel. The staple cartridge comprises staples removably stored therein, a first component comprising a first key, and a second component comprising a second key. The staples are deployable from the staple cartridge based on the firing driver moving toward the distal position. The first key is configured to place the first lockout in the first unlocked state based on the staple cartridge being positioned in the elongate channel. The second key is configured to place the second lockout in the second unlocked state based on the staple cartridge being positioned in the elongate channel.

Example 32—The surgical stapling system of Example 31, wherein the first component comprises a cartridge body, and wherein the second component comprises a sled.

Example 33—A surgical stapling system comprising an end effector, a firing actuator movable from a proximal position toward a distal position during a firing stroke, a first lockout, and a second lockout. The end effector comprises a first jaw and a second jaw pivotable relative to the first jaw between an open position and a closed position. The firing actuator defines a first notch and a second notch. The first lockout is configurable in a first locked state in which the first lockout is received in the first notch and a first unlocked state in which the first lockout is removed from the first notch. A staple cartridge comprising a first key is configured to place the first lockout in the first unlocked state based on the staple cartridge being positioned in the end effector. The second lockout is configurable in a second locked state in which the second lockout is received in the second notch and a second unlocked state in which the second lockout is removed from the second notch. A staple cartridge comprising a second key is configured to place the second lockout in the second unlocked state based on the staple cartridge being positioned in the end effector.

Example 34—The surgical stapling system of Example 33, wherein the firing actuator comprises a firing driver and a firing bar extending from the firing driver. The first notch and the second notch are defined in the firing driver.

Example 35—The surgical stapling system of Example 33, wherein the firing actuator comprises a firing driver, a first push rod operably coupled to an upper end of the firing driver, and a second push rod operably coupled to a lower end of the firing driver. The first notch is defined in the first push rod. The second notch is defined in the second firing rod.

The entire disclosures of U.S. Pat. No. 11,589,865, entitled METHODS FOR CONTROLLING A POWERED SURGICAL STAPLER THAT HAS SEPARATE ROTARY CLOSURE AND FIRING SYSTEMS, which issued on Feb. 28, 2023, U.S. Pat. No. 6,978,921, entitled SURGICAL STAPLING INSTRUMENT INCORPORATING AN E-BEAM FIRING MECHANISM, which issued on Dec. 27, 2005, U.S. Pat. No. 10,213,203, entitled STAPLE CARTRIDGE ASSEMBLY WITHOUT A BOTTOM COVER, which issued on Feb. 26, 2019, U.S. Pat. No. 10,945,727, entitled STAPLE CARTRIDGE WITH DEFORMABLE DRIVER RETENTION FEATURES, which issued on Mar. 16, 2021, U.S. Pat. No. 11,234,698, entitled STAPLING SYSTEM COMPRISING A CLAMP LOCKOUT AND A FIRING LOCKOUT, which issued on Feb. 1, 2022, U.S. Pat. No. 11,540,826, entitled SURGICAL STAPLER END EFFECTOR SLED HAVING CARTRIDGE WALL SUPPORT FEATURE, which issued on Jan. 3, 2023, U.S. Pat. No. 10,299,792, entitled FASTENER CARTRIDGE COMPRISING NON-UNIFORM FASTENERS, which issued on May 28, 2019, U.S. Pat. No. 8,540,133, entitled STAPLE CARTRIDGE, which issued on Sep. 24, 2013, U.S. Pat. No. 9,788,835, entitled DEVICES AND METHODS FOR FACILITATING EJECTION OF SURGICAL FASTENERS FROM CARTRIDGES, which issued on Oct. 17, 2017, U.S. Pat. No. 10,105,142, entitled SURGICAL STAPLER WITH PLURALITY OF CUTTING ELEMENTS, which issued on Oct. 23, 2018, U.S. Pat. No. 10,537,324, entitled STEPPED STAPLE CARTRIDGE WITH ASYMMETRICAL STAPLES, which issued on Jan. 21, 2020, U.S. Pat. No. 7,669,746, entitled STAPLE CARTRIDGES FOR FORMING STAPLES HAVING DIFFERING FORMED STAPLE HEIGHTS, which issued on Mar. 2, 2010, U.S. Pat. No. 8,123,100, entitled SURGICAL STAPLING INSTRUMENTS INCLUDING A CARTRIDGE HAVING MULTIPLE STAPLE SIZES, which issued on Feb. 28, 2012, U.S. Pat. No. 7,407,075, entitled STAPLE CARTRIDGE HAVING MULTIPLE STAPLE SIZES FOR A SURGICAL STAPLING INSTRUMENT, which issued on Aug. 5, 2008, U.S. Pat. No. 10,085,749, entitled SURGICAL APPARATUS WITH CONDUCTOR STRAIN RELIEF, which issued on Oct. 2, 2018, U.S. Pat. No. 10,765,427, entitled METHOD FOR ARTICULATING A SURGICAL INSTRUMENT, which issued on Sep. 8, 2020, U.S. Pat. No. 11,291,445, entitled SURGICAL STAPLE CARTRIDGES WITH INTEGRAL AUTHENTICATION KEYS, which issued on Apr. 5, 2022, U.S. Pat. No. 8,864,007, entitled IMPLANTABLE FASTENER CARTRIDGE HAVING A NON-UNIFORM ARRANGEMENT, which issued on Oct. 21, 2014, U.S. Pat. No. 11,490,890, entitled COMPRESSIBLE NON-FIBROUS ADJUNCTS, which issued on Nov. 8, 2022, U.S. Pat. No. 10,952,724, entitled THREE DIMENSIONAL ADJUNCTS, which issued on Mar. 23, 2021, U.S. Pat. No. 9,770,245, entitled LAYER ARRANGEMENTS FOR SURGICAL STAPLE CARTRIDGES, which issued on Sep. 26, 2017, U.S. Pat. No. 10,123,798, entitled TISSUE THICKNESS COMPENSATOR COMPRISING CONTROLLED RELEASE AND EXPANSION, which issued on Nov. 13, 2018, U.S. Pat. No. 10,166,023, entitled METHOD OF APPLYING A BUTTRESS TO A SURGICAL STAPLER END EFFECTOR, which issued on Jan. 1, 2019, U.S. Pat. No. 11,207,065, entitled METHOD FOR FABRICATING SURGICAL STAPLER ANVILS, which issued on Dec. 28, 2021, U.S. Pat. No. 8,141,762, entitled SURGICAL STAPLER COMPRISING A STAPLE POCKET, which issued on Mar. 27, 2012, U.S. Pat. No. 8,876,857, entitled END EFFECTOR WITH REDUNDANT CLOSING MECHANISMS, which issued on Nov. 4, 2014, U.S. Pat. No. 9,629,631, entitled COMPOSITE DRIVE BEAM FOR SURGICAL STAPLING, which issued on Apr. 25, 2017, U.S. Patent Application Publication No. 2022/0346858, entitled METHOD FOR OPERATING A SURGICAL INSTRUMENT INCLUDING SEGMENTED ELECTRODES, which published on Nov. 3, 2022, U.S. Patent Application Publication No. 2022/0304680, entitled DRIVERS FOR FASTENER CARTRIDGE ASSEMBLIES HAVING ROTARY DRIVE SCREWS, which published on Sep. 29, 2022, U.S. Patent Application Publication No. 2022/0304679, entitled METHOD OF USING A POWERED STAPLING DEVICE, which published on Sep. 29, 2022, U.S. Patent Publication No. 2019/0298350, entitled METHODS FOR CONTROLLING A POWERED SURGICAL STAPLER THAT HAS SEPARATE ROTARY CLOSURE AND FIRING SYSTEMS, which published on Oct. 3, 2019, U.S. Patent Application Publication No. 2017/0367695, entitled STAPLE CARTRIDGE COMPRISING WIRE STAPLES AND STAMPED STAPLES, which published on Dec. 28, 2017, U.S. Patent Application Publication No. 2015/0134077, entitled SEALING MATERIALS FOR USE IN SURGICAL STAPLING, which published on May 14, 2015, U.S. Patent Application Publication No. 2018/0168615, entitled METHOD OF DEFORMING STAPLES FROM TWO DIFFERENT TYPES OF STAPLE CARTRIDGES WITH THE SAME SURGICAL STAPLING INSTRUMENT, which published on Jun. 21, 2018, U.S. Patent Application Publication No. 2018/0132849, entitled STAPLE FORMING POCKET CONFIGURATIONS FOR CIRCULAR SURGICAL STAPLER ANVIL, which published on May 17, 2018, U.S. Patent Application Publication No. 2018/0168613, entitled SURGICAL INSTRUMENTS WITH JAWS THAT ARE PIVOTABLE ABOUT A FIXED AXIS AND INCLUDE SEPARATE AND DISTINCT CLOSURE AND FIRING SYSTEMS, which published on Jun. 21, 2018, U.S. Patent Application Publication No. 2017/0319205, entitled POWERED END EFFECTOR ASSEMBLY WITH PIVOTABLE CHANNEL, which published on Nov. 9, 2017, U.S. Patent Application Publication No. 2014/0001231, entitled FIRING SYSTEM LOCKOUT ARRANGEMENTS FOR SURGICAL INSTRUMENTS, which published on Jan. 2, 2014, U.S. Patent Application Publication No. 2016/0095596, entitled APPARATUS FOR ENDOSCOPIC PROCEDURES, which published on Apr. 7, 2016, U.S. Patent Application Publication No. 2015/0297199, entitled ADAPTER ASSEMBLY WITH GIMBAL FOR INTERCONNECTING ELECTROMECHANICAL SURGICAL DEVICES AND SURGICAL LOADING UNITS, AND SURGICAL SYSTEMS THEREOF, which published on Oct. 22, 2015, U.S. Patent Application Publication No. 2022/0031351, entitled SURGICAL INSTRUMENTS WITH DIFFERENT ARTICULATION JOINT ARRANGEMENTS FOR ACCOMMODATING FLEXIBLE ACTUATORS, which published on Feb. 3, 2022, U.S. Patent Application Publication No. 2022/0031320, entitled SURGICAL INSTRUMENTS WITH FLEXIBLE FIRING MEMBER ACTUATOR CONSTRAINT ARRANGEMENTS, which published on Feb. 3, 2022, U.S. Patent Application Publication No. 2023/0119119, entitled CABLE-DRIVEN ACTUATION SYSTEM FOR ROBOTIC SURGICAL TOOL ATTACHMENT, which published on Apr. 20, 2023, International Patent Publication No. WO2018/071497, entitled STAPLER CARTRIDGE WITH AN INTEGRAL KNIFE, which published on Apr. 18, 2018, International Patent Publication No. WO2018/049211, entitled WRIST ARCHITECTURE, which published on Mar. 15, 2018, U.S. Pat. No. 11,298,129, entitled METHOD FOR PROVIDING AN AUTHENTICATION LOCKOUT IN A SURGICAL STAPLER WITH A REPLACEABLE CARTRIDGE, which issued on Apr. 12, 2022, U.S. Pat. No. 10,898,183, entitled ROBOTIC SURGICAL INSTRUMENT WITH CLOSED LOOP FEEDBACK TECHNIQUES FOR ADVANCEMENT OF CLOSURE MEMBER DURING FIRING, which issued on Jan. 26, 2021, U.S. Pat. No. 5,485,947, entitled LINEAR STAPLING MECHANISM WITH CUTTING MEANS, which issued on Jan. 23, 1996, International Patent Publication No. WO2018/049206, entitled STAPLER RELOAD DETECTION AND IDENTIFICATION, which published on Mar. 15, 2018, U.S. Patent Application Publication No. 2016/0249920, entitled Surgical fastener applying apparatus, which published on Sep. 1, 2016, U.S. Design Patent No. D974,560, entitled STAPLE CARTRIDGE, which issued on Jan. 3, 2023, U.S. Design Patent No. D967,421, entitled STAPLE CARTRIDGE, which issued on Oct. 18, 2022, U.S. Design Patent No. D933,220, entitled BUTTRESS ASSEMBLY FOR A SURGICAL STAPLER, which issued on Oct. 12, 2021, U.S. Pat. No. 9,839,420, entitled TISSUE THICKNESS COMPENSATOR COMPRISING AT LEAST ONE MEDICAMENT, which issued on Dec. 12, 2017, U.S. Pat. No. 10,588,623, entitled ADHESIVE FILM LAMINATE, which issued on Mar. 17, 2020, U.S. Pat. No. 8,499,992, entitled DEVICE AND METHOD FOR CONTROLLING COMPRESSION OF TISSUE, which issued on Aug. 6, 2013, U.S. Patent Application Publication No. 2022/0378427, entitled STAPLING INSTRUMENT COMPRISING JAW MOUNTS, which published on Dec. 1, 2022, U.S. Pat. No. 10,349,939, entitled METHOD OF APPLYING A BUTTRESS TO A SURGICAL STAPLER, which issued on Jul. 16, 2019, U.S. Pat. No. 9,386,988, entitled RETAINER ASSEMBLY INCLUDING A TISSUE THICKNESS COMPENSATOR, which issued on Jul. 12, 2016, U.S. Pat. No. 9,072,535, entitled SURGICAL STAPLING INSTRUMENTS WITH ROTATABLE STAPLE DEPLOYMENT ARRANGEMENTS, which issued on Jul. 7, 2015, and U.S. Pat. No. 9,844,369, entitled, SURGICAL END EFFECTORS WITH FIRING ELEMENT MONITORING ARRANGEMENTS, which issued on Dec. 19, 2017 are incorporated by reference herein.

The entire disclosures of:
U.S. Pat. No. 5,403,312, entitled ELECTROSURGICAL HEMOSTATIC DEVICE, which issued on Apr. 4, 1995;
U.S. Pat. No. 7,000,818, entitled SURGICAL STAPLING INSTRUMENT HAVING SEPARATE DISTINCT CLOSING AND FIRING SYSTEMS, which issued on Feb. 21, 2006;
U.S. Pat. No. 7,422,139, entitled MOTOR-DRIVEN SURGICAL CUTTING AND FASTENING INSTRUMENT WITH TACTILE POSITION FEEDBACK, which issued on Sep. 9, 2008;
U.S. Pat. No. 7,464,849, entitled ELECTRO-MECHANICAL SURGICAL INSTRUMENT WITH CLOSURE SYSTEM AND ANVIL ALIGNMENT COMPONENTS, which issued on Dec. 16, 2008;
U.S. Pat. No. 7,670,334, entitled SURGICAL INSTRUMENT HAVING AN ARTICULATING END EFFECTOR, which issued on Mar. 2, 2010;
U.S. Pat. No. 7,753,245, entitled SURGICAL STAPLING INSTRUMENTS, which issued on Jul. 13, 2010;
U.S. Pat. No. 8,393,514, entitled SELECTIVELY ORIENTABLE IMPLANTABLE FASTENER CARTRIDGE, which issued on Mar. 12, 2013;
U.S. patent application Ser. No. 11/343,803, entitled SURGICAL INSTRUMENT HAVING RECORDING CAPABILITIES, now U.S. Pat. No. 7,845,537;
U.S. patent application Ser. No. 12/031,573, entitled SURGICAL CUTTING AND FASTENING INSTRUMENT HAVING RF ELECTRODES, filed Feb. 14, 2008;
U.S. patent application Ser. No. 12/031,873, entitled END EFFECTORS FOR A SURGICAL CUTTING AND STAPLING INSTRUMENT, filed Feb. 15, 2008, now U.S. Pat. No. 7,980,443;
U.S. patent application Ser. No. 12/235,782, entitled MOTOR-DRIVEN SURGICAL CUTTING INSTRUMENT, now U.S. Pat. No. 8,210,411;
U.S. patent application Ser. No. 12/235,972, entitled MOTORIZED SURGICAL INSTRUMENT, now U.S. Pat. No. 9,050,083.
U.S. patent application Ser. No. 12/249,117, entitled POWERED SURGICAL CUTTING AND STAPLING APPARATUS WITH MANUALLY RETRACTABLE FIRING SYSTEM, now U.S. Pat. No. 8,608,045;
U.S. patent application Ser. No. 12/647, 100, entitled MOTOR-DRIVEN SURGICAL CUTTING INSTRUMENT WITH ELECTRIC ACTUATOR DIRECTIONAL CONTROL ASSEMBLY, filed Dec. 24, 2009, now U.S. Pat. No. 8,220,688;
U.S. patent application Ser. No. 12/893,461, entitled STAPLE CARTRIDGE, filed Sep. 29, 2012, now U.S. Pat. No. 8,733,613;
U.S. patent application Ser. No. 13/036,647, entitled SURGICAL STAPLING INSTRUMENT, filed Feb. 28, 2011, now U.S. Pat. No. 8,561,870;
U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535;
U.S. patent application Ser. No. 13/524,049, entitled ARTICULATABLE SURGICAL INSTRUMENT COMPRISING A FIRING DRIVE, filed on Jun. 15, 2012, now U.S. Pat. No. 9,101,358;
U.S. patent application Ser. No. 13/800,025, entitled STAPLE CARTRIDGE TISSUE THICKNESS SENSOR SYSTEM, filed on Mar. 13, 2013, now U.S. Pat. No. 9,345,481;
U.S. patent application Ser. No. 13/800,067, entitled STAPLE CARTRIDGE TISSUE THICKNESS SENSOR SYSTEM, filed on Mar. 13, 2013, now U.S. Patent Application Publication No. 2014/0263552;
U.S. Patent Application Publication No. 2007/0175955, entitled SURGICAL CUTTING AND FASTENING INSTRUMENT WITH CLOSURE TRIGGER LOCKING MECHANISM, filed Jan. 31, 2006; and
U.S. Patent Application Publication No. 2010/0264194, entitled SURGICAL STAPLING INSTRUMENT WITH AN ARTICULATABLE END EFFECTOR, filed Apr. 22, 2010, now U.S. Pat. No. 8,308,040, are hereby incorporated by reference herein.

While several configurations have been described, additional modifications are within the scope of the present disclosure, which is intended to cover any variations, uses, or adaptations of the disclosed configurations using its general principles.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

It is worthy to note that any reference to “one aspect,” “an aspect,” “an exemplification,” “one exemplification,” and the like means that a particular feature, structure, or characteristic described in connection with the aspect is included in one aspect. Thus, appearances of the phrases “in one aspect,” “in an aspect,” “in an exemplification,” and “in one exemplification” in various places throughout the specification are not necessarily all referring to the same aspect. The particular features, structures or characteristics may be combined in any suitable manner in various aspects.

It is worthy to note that any reference numbers included in the appended claims are used to reference exemplary embodiments/elements described in the present disclosure. Accordingly, any such reference numbers are not meant to limit the scope of the subject matter recited in the appended claims.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

1-15. (canceled)

16. A surgical stapling system, comprising:
an end effector, comprising:
a first jaw; and
a second jaw pivotable relative to the first jaw between an open position and a closed position;
a firing actuator movable from a proximal position toward a distal position during a firing stroke, wherein the firing stroke comprises a first stroke portion and a second stroke portion distal to the first stroke portion, and wherein the firing actuator comprises a first edge and a second edge;
a first lockout configurable in:
a first locked state in which the first lockout engages the first edge during the firing stroke; and
a first unlocked state in which the first edge bypasses the first lockout during the firing stroke;
wherein a staple cartridge comprising a first key is configured to place the first lockout in the first unlocked state based on the staple cartridge being positioned in the end effector; and
a second lockout configurable in:
a second locked state in which the second lockout engages the second edge during the firing stroke; and
a second unlocked state in which the second edge bypasses the second lockout during the firing stroke;
wherein a staple cartridge comprising a second key is configured to place the second lockout in the second unlocked state based on the staple cartridge being positioned in the end effector.

17. The surgical stapling system of claim 16, wherein the firing actuator is prevented from moving through the firing stroke absent a staple cartridge comprising the first key and the second key being positioned in the end effector.

18. The surgical stapling system of claim 16, wherein the firing actuator is permitted to move through the first stroke portion but prevented from moving through the second stroke portion based on a staple cartridge being positioned in the end effector that comprises the first key but does not comprise the second key.

19. The surgical stapling system of claim 18, wherein the firing actuator comprises a sidewall, wherein the second lockout is configured to engage the sidewall to maintain the second lockout in the second unlocked state during the first stroke portion.

20. The surgical stapling system of claim 18, wherein the firing actuator is permitted to move through the first stroke portion with the second lockout being in the second locked state.

21. The surgical stapling system of claim 16, wherein:
the first edge is a first length from a distal end thereof;
the second edge is a second length from the distal end thereof; and
the first length is the same as the second length.

22. The surgical stapling system of claim 16, wherein:
the first edge is a first length from a distal end thereof;
the second edge is a second length from the distal end thereof; and
the second length is different than the first length.

23. The surgical stapling system of claim 16, wherein:
the firing actuator defines a first notch and a second notch;
the first notch comprises the first edge and a first sidewall extending from the first edge;
the second notch comprises the second edge and a second sidewall extending from the second edge;
the first sidewall comprises a first width;
the second sidewall comprises a second width; and
the second width is the same as the first width.

24. The surgical stapling system of claim 16, wherein:
the firing actuator defines a first notch and a second notch;
the first notch comprises the first edge and a first sidewall extending from the first edge;
the second notch comprises the second edge and a second sidewall extending from the second edge;
the first sidewall comprises a first width;
the second sidewall comprises a second width; and
the second width is different than the first width.

25. The surgical stapling system of claim 16, wherein the firing actuator comprises a firing driver and a firing bar extending from the firing driver, and wherein the firing driver comprises the first edge and the second edge.

26. The surgical stapling system of claim 16, wherein the firing actuator comprises:
a firing driver;
a first push rod operably coupled to an upper end of the firing driver, wherein the first push rod comprises the first edge; and
a second push rod operably coupled to a lower end of the firing driver, wherein the second push rod comprises the second edge.

27. The surgical stapling system of claim 16, wherein:
the first lockout comprises a first wedge and a first spring configured to bias the first wedge toward the first edge; and
the second lockout comprises a second wedge and a second spring configured to bias the second wedge toward the second edge.

28. The surgical stapling system of claim 27, wherein:
the first wedge comprises a first cam surface, wherein the first key is configured to engage the first cam surface to move the first wedge away from the first edge; and
the second wedge comprises a second cam surface, wherein the second key is configured to engage the second cam surface to move the second wedge away from the second edge.

29. The surgical stapling system of claim 16, wherein:
the first lockout comprises:
a first lockout arm pivotably coupled to the first jaw; and
a first spring configured to bias the first lockout arm toward the first edge; and
the second lockout comprises:
a second lockout arm pivotably coupled to the first jaw; and
a second spring configured to bias the second lockout arm toward the second edge.

30. The surgical stapling system of claim 29, wherein:
the first lockout arm comprises a first cam surface, wherein the first key is configured to engage the first cam surface to rotate the first lockout arm away from the first edge; and
the second lockout arm comprises a second cam surface, wherein the second key is configured to engage the second cam surface to rotate the second lockout arm away from the second edge.

31. A surgical stapling system, comprising:
an end effector, comprising:
an elongate channel; and
an anvil pivotable relative to the elongate channel between an open position and a closed position;
a firing driver movable from a proximal position toward a distal position during a firing stroke, wherein the firing driver comprises a first shoulder and a second shoulder;
a first lockout, configurable between:
a first locked state in which the first lockout engages the first shoulder during the firing stroke; and
a first unlocked state in which the first shoulder bypasses the first lockout during the firing stroke;
a second lockout, configurable between:
a second locked state in which the second lockout engages the second shoulder during the firing stroke; and
a second unlocked state in which the second shoulder bypasses the second lockout during the firing stroke; and
a staple cartridge configured to be removably positioned in the elongate channel, wherein the staple cartridge comprises:
staples removably stored therein, wherein the staples are deployable from the staple cartridge based on the firing driver moving toward the distal position;
a first component comprising a first key, wherein the first key is configured to place the first lockout in the first unlocked state based on the staple cartridge being positioned in the elongate channel; and
a second component comprising a second key, wherein the second key is configured to place the second lockout in the second unlocked state based on the staple cartridge being positioned in the elongate channel.

32. The surgical stapling system of claim 31, wherein the first component comprises a cartridge body, and wherein the second component comprises a sled.

33. A surgical stapling system, comprising:
an end effector, comprising:
a first jaw; and
a second jaw pivotable relative to the first jaw between an open position and a closed position;
a firing actuator movable from a proximal position toward a distal position during a firing stroke, and wherein the firing actuator defines a first notch and a second notch;
a first lockout configurable in:
a first locked state in which the first lockout is received in the first notch; and
a first unlocked state in which the first lockout is removed from the first notch;
wherein a staple cartridge comprising a first key is configured to place the first lockout in the first unlocked state based on the staple cartridge being positioned in the end effector; and
a second lockout configurable in:
a second locked state in which the second lockout is received in the second notch; and
a second unlocked state in which the second lockout is removed from the second notch;
wherein a staple cartridge comprising a second key is configured to place the second lockout in the second unlocked state based on the staple cartridge being positioned in the end effector.

34. The surgical stapling system of claim 33, wherein the firing actuator comprises a firing driver and a firing bar extending from the firing driver, and wherein the first notch and the second notch are defined in the firing driver.

35. The surgical stapling system of claim 33, wherein the firing actuator comprises:
a firing driver;
a first push rod operably coupled to an upper end of the firing driver, wherein the first notch is defined in the first push rod; and
a second push rod operably coupled to a lower end of the firing driver, wherein the second notch is defined in the second firing rod.
